(19) United States
(12) Patent Application Publication
(10) Pub. No.: US 2026/0191192 A1
(43) Pub. Date: Jul. 9, 2026

(54) ANTIMICROBIAL COMPOSITIONS COMPRISING PELARGONIC ACID

(71) Applicant: LANXESS Corporation, Pittsburgh, PA (US)

(72) Inventor: Ziang Li, Newark, DE (US)

(21) Appl. No.: 19/522,031

(22) PCT Filed: May 17, 2024
(86) PCT No.: PCT/US2024/029933

§ 371 Date: Mar. 23, 2026

Related U.S. Application Data
(60) Provisional application No. 63/586,437, filed on Sep. 29, 2023.
(60) Provisional application No. 63/562,987, filed on Mar. 8, 2024.

(51) Int. Cl. A01N 31/14; A01N 25/02; A01N 25/22; A01N 31/04; A01P 1/00; A61K 8/34; A61K 8/36; A61Q 17/00

(52) U.S. Cl. CPC A01N 31/14; A01N 25/02; A01N 25/22; A01N 31/04; A01P 1/00; A61K 8/34; A61K 8/361; A61Q 17/005

(57) ABSTRACT

The present disclosure provides antimicrobial compositions containing phenoxyethanol, benzyl alcohol or a combination thereof as an antimicrobial agent and pelargonic acid and/or a salt thereof and further provides uses and applications of the presently disclosed antimicrobial compositions in product formulations, particularly personal care formulations and home care formulations.

### FIELD OF INVENTION

The present invention relates to synergistically enhancing the antimicrobial activity of certain antimicrobial agents useful in a wide range of product formulations, particularly personal care and home care formulations.

### BACKGROUND OF THE INVENTION

Preservation against microbial contamination of product formulations, such as personal care products (e.g., cosmetic products) and home care products, plays a pivotal role in consumer safety, product stability, integrity, and quality assurance, regulatory compliance, sustainability, among other factors. Traditional preservatives, such as isothiazolinones, formaldehyde releasers, parabens, and IPBC, are under increasing scrutiny concerning potential health and environmental impacts. This, along with consumer preferences, have led to a growing demand for alternative preservation agents that are both effective and aligned with contemporary expectations. While many product formulators have turned to “soft” preservatives, such as phenoxyethanol and benzyl alcohol, such agents alone, particularly at desired dosing levels, often do not provide full spectrum antimicrobial efficacy necessary for robust protection, potentially complicating their use in product formulations and/or requiring undesirably high dosing levels.

Accordingly, there is a pressing need to address the efficacy gap of certain soft preservatives, while aligning with increasing consumer demand for natural and sustainable products.

This need and others are met by the present disclosure as described further herein.

### SUMMARY OF INVENTION

The present disclosure provides an antimicrobial composition comprising (a) an antimicrobial agent selected from the group consisting of phenoxyethanol, benzyl alcohol and combinations thereof, and (b) pelargonic acid and/or a salt thereof, wherein the ratio by weight of the antimicrobial agent (a) to the pelargonic acid (b) is from about 40:1 to about 1:5, preferably from about 40:1 or from about 20:1 to about 1:2.5.

Also provided is an aqueous product formulation, such as a personal care or home care product formulation, comprising an antimicrobial effective amount of the antimicrobial composition of the present disclosure.

Further provided is a method of controlling or inhibiting the growth of microorganisms in a product formulation, such as a personal care or home care formulation, comprising adding to the formulation (a) an antimicrobial agent selected from the group consisting of phenoxyethanol, benzyl alcohol and combinations thereof, and (b) pelargonic acid and/or a salt thereof, wherein the ratio by weight of the antimicrobial agent (a) to the pelargonic acid and/or salt thereof (b) is from about 40:1 to about 1:5, preferably from about 40:1 or from about 20:1 to about 1:2.5, and the antimicrobial agent (a) and the pelargonic acid and/or salt thereof (b) are added in an antimicrobial effective amount.

In a further aspect, the present disclosure includes a method of enhancing the antimicrobial efficacy of an antimicrobial agent selected from the group consisting of phenoxyethanol, benzyl alcohol and combinations thereof, comprising combining or mixing pelargonic acid and/or a salt thereof with the antimicrobial agent in a ratio by weight of the antimicrobial agent to the pelargonic acid of from about 40:1 to about 1:5, preferably from about 40:1 or from about 20:1 to about 1:2.5.

Also provided is the use of pelargonic acid and/or a salt thereof to enhance the antimicrobial efficacy of an antimicrobial agent selected from the group consisting of phenoxyethanol, benzyl alcohol and combinations thereof, wherein the ratio by weight of the antimicrobial agent to the pelargonic acid and/or salt thereof is from about 40:1 to about 1:5, preferably from about 40:1 or from about 20:1 to about 1:2.5.

Additionally provided is a method of reducing the minimum amount of an antimicrobial preservative needed for preservation activity in a product formulation, such as a personal care or home care formulation, the method comprising combining or mixing pelargonic acid and/or a salt thereof with the antimicrobial agent in the product formulation, wherein the antimicrobial agent is selected from the group consisting of phenoxyethanol, benzyl alcohol and combinations thereof, and the ratio by weight of the antimicrobial agent to the pelargonic acid and/or salt thereof is from about 40:1 to about 1:5, preferably from about 40:1 or from about 20:1 to about 1:2.5.

### DETAILED DESCRIPTION

In accordance with the present disclosure, it was surprisingly discovered that pelargonic acid and/or a salt thereof, when combined at the ratios described herein with an antimicrobial agent selected from phenoxyethanol, benzyl alcohol and combinations thereof, synergistically enhances the antimicrobial activity of the antimicrobial agent. Moreover, as demonstrated in the present disclosure, it was further unexpectedly found that the enhanced antimicrobial activity of the antimicrobial agent when combined with pelargonic acid and/or a salt thereof is achieved in product formulations having a pH above (even well above) the pKa of pelargonic acid (pKa of 4.95 at 25° C.), such as in formulations having a pH of at least 6.0, at least 6.5, at least 7.0 or higher, as described herein. The presently disclosed combination with pelargonic acid is therefore advantageously useful in an expansive scope of product formulations, while additionally allowing for a reduction in the amount of the antimicrobial agent typically required to achieve sufficient antimicrobial performance. Furthermore, pelargonic acid and/or a salt thereof and the antimicrobial agent selected from phenoxyethanol, benzyl alcohol and combinations thereof can be combined in an antimicrobial composition, such as for use in inhibiting microbial growth in or preserving an end product formulation, without requiring the presence of an added solvent in the antimicrobial composition. In accordance with the present disclosure, it was further discovered that an additional antimicrobial agent, such as an alkylhydroxamic acid, can be used, typically in a minority amount relative to the combined amount of the antimicrobial agent (a) and the pelargonic acid and/or salt thereof (b), to increase the antimicrobial performance of the composition at the same total load level. As demonstrated herein, pelargonic acid and/or a salt thereof shows superior enhancement of the antimicrobial activity of the antimicrobial agent as compared to two commercial multifunctional booster benchmarks 2-ethyl-hexyl glycerin and 1,2-hexanediol at the same dosing level.

In addition, it was further found in accordance with the present disclosure that the stability over time of an antimicrobial composition comprising (a) an antimicrobial agent selected from the group consisting of phenoxyethanol, benzyl alcohol and combinations thereof and (b) pelargonic acid and/or a salt thereof can be significantly enhanced via the inclusion of at least one alkaline stabilizer.

Unless otherwise specified, the following terms are defined as follows:

As used herein, “formulation” refers to a preparation that is to be preserved or provided with antimicrobial activity by employing the combination of the antimicrobial agent (a) and the pelargonic acid and/or salt thereof (b) of the present disclosure.

As used herein, an “antimicrobial effective amount” means an amount to provide a desired antimicrobial effect or activity, such as a desired preservative, antibacterial and/or antifungal effect.

As used herein, “synergistic” and the like mean the antimicrobial effect (e.g., antibacterial and/or antifungal effect) of the combination is greater than the additive effect of each component.

As used herein, “microorganism” includes, for example, bacteria and fungi (such as yeast and mold).

pH values or ranges thereof refer to pH at room temperature (20-25° C.), unless otherwise specified.

The antimicrobial composition of the present disclosure comprises (a) an antimicrobial agent selected from the group consisting of phenoxyethanol (2-phenoxyethan-1-ol), benzyl alcohol and combinations thereof, and (b) pelargonic acid and/or a salt thereof, wherein the ratio by weight of the antimicrobial agent (a) to the pelargonic acid or salt thereof (b) is from about 40:1 to about 1:5, preferably from about 40:1 or from about 20:1 to about 1:2.5. Pelargonic acid (also known as nonanoic acid) is a naturally occurring fatty acid with a saturated nine-carbon straight chain and can be naturally derived from plant oils, among other extraction or production processes. Examples of suitable salts of pelargonic acid include, but are not limited to, alkali salts, such as sodium and potassium (i.e., sodium nonanoate, potassium nonanoate), and ammonium salt (i.e., ammonium nonanoate).

In many embodiments, the antimicrobial agent (a) is phenoxyethanol. In many other embodiments, the antimicrobial agent (a) is benzyl alcohol. In further embodiments, the antimicrobial agent (a) is any combination of phenoxyethanol and benzyl alcohol.

Preferably, the ratio by weight of the antimicrobial agent (a) to the pelargonic acid and/or salt thereof (b) is from about 40:1 or from about 20:1 to about 1:2.5, such as from about 20:1, preferably from about 15:1, more preferably from about 10:1, even more preferably from about 5:1 or from about 3:1 to about 1:2, preferably to about 1:1.5, to about 1:1.25, to about 1:1.2, to about 1:1.15, to about 1:1.1, to about 1:1.05 or more preferably to about 1:1. In particular, the ratio is preferably from about 15:1 to about 1:1.5, more preferably from about 10:1, even more preferably from about 5:1 or from about 3:1 to about 1:1.25, to about 1:1.2, to about 1:1.1, or more preferably to about 1:1. Optimized amounts and ratios may vary depending, for example, on the application, the desired properties of the product, and the like. As demonstrated herein, the antimicrobial activity (e.g., an antibacterial and/or antifungal effect) of the antimicrobial agent (a) is synergistically enhanced against various microorganisms, including bacteria and fungi. For example, the presently disclosed combination preferably controls gram positive bacteria, gram negative bacteria, and fungi, among other microorganisms. Non-limiting examples include Staphylococcus aureus, Staphylococcus epidermidis, Pseudomonas aeruginosa, Escherichia coli, Enterobacter gergoviae, Klebsiella pneumoniae, Burholderia cepacia, Pseudomonas putida, Candida albicans, Aspergillus brasiliensis, and mixtures thereof.

The antimicrobial agent (a) and the pelargonic acid and/or salt thereof (b) typically collectively constitute 50 to 100 wt % of the active ingredients in the antimicrobial composition, often a majority (>50 wt %) of the active ingredients, such as at least 60 wt % or at least 70 wt %, more preferably at least 80 wt %, even more preferably at least 85 wt %, at least 90 wt %, at least 95 wt %, at least 96 wt %, at least 97 wt %, at least 98 wt % or at least 99 wt %, based on the total weight of active ingredients in the antimicrobial composition. The antimicrobial composition often contains from about 60 to 100 wt %, preferably from about 65 to 100 wt %, from about 70 to 100 wt %, from about 75 to 100 wt %, from about 80 to 100 wt %, from about 85 to 100 wt %, from about 90 to 100 wt %, or from about 95 or from about 97 to 100 wt %, of the antimicrobial agent (a) and the pelargonic acid and/or salt thereof (b) combined, based on the total weight of active ingredients in the antimicrobial composition. In many embodiments, the antimicrobial composition comprises active ingredients consisting essentially of, or in further embodiments consisting of, the antimicrobial agent (a) and the pelargonic acid and/or salt thereof (b).

One or more than one additional antimicrobial agent (c), such as antimicrobial agents suitable for use in personal care formulations or home care formulations, may be present in the antimicrobial composition, and, if present, preferably constitutes no greater than 50 wt % of the active ingredients in the antimicrobial composition, typically a minority (<50 wt %) of the active ingredients, such as no greater than 40 wt % or no greater than 30 wt %, more preferably no greater than 25 wt %, no greater than 20 wt %, even more preferably no greater than 15 wt %, no greater than 10 wt %, no greater than 5 wt %, or no greater than 3 wt %, based on the total weight of active ingredients in the antimicrobial composition.

Preferably, the antimicrobial composition contains from about 70 to 100 wt %, from about 75 to 100 wt %, from about 80 to 100 wt %, from about 85 to 100 wt %, from about 90 to 100 wt %, or from about 95 or from about 97 to 100 wt %, of the antimicrobial agent (a), the pelargonic acid and/or salt thereof (b) and the one or more than one additional antimicrobial agent (c) combined, based on the total weight of active ingredients in the antimicrobial composition. Preferably, the antimicrobial agent (a), the pelargonic acid and/or salt thereof (b) and the one or more than one additional antimicrobial agent (c) constitute all or substantially all of the active ingredients in the antimicrobial composition. In many embodiments, the antimicrobial composition comprises active ingredients consisting essentially of, or in further embodiments consisting of, the antimicrobial agent (a), the pelargonic acid and/or salt thereof (b) and the one or more than one additional antimicrobial agent (c).

The antimicrobial agent (a) and the pelargonic acid and/or salt thereof (b) typically collectively constitute from about 50 to about 100 wt % of the total weight of the antimicrobial composition, often a majority (>50 wt %) of the antimicrobial composition, such as at least 60 wt %, preferably at least 70 wt %, more preferably at least 75 wt %, at least 80 wt %, even more preferably at least 85 wt %, or at least 90 wt %, based on the total weight of the antimicrobial composition. The antimicrobial composition often contains the antimicrobial agent (a) and the pelargonic acid and/or salt thereof (b) in a combined amount of from about 60 to 100 wt %, preferably from about 70 to 100 wt %, from about 75 to 100 wt %, from about 80 to 100 wt %, more preferably from about 85 to 100 wt %, or from about 90 to 100 wt %, or from about 60 wt %, preferably from about 70 wt %, more preferably from about 75 wt %, from about 80 wt % or from about 85 wt % to about 99 wt %, to about 97 wt %, to about 95 wt % or to about 92 wt %, based on the total weight of the antimicrobial composition. In many embodiments, the antimicrobial composition consists essentially of, or in further embodiments consists of, the antimicrobial agent (a) and the pelargonic acid and/or salt thereof (b).

Where the antimicrobial composition includes the one or more than one additional antimicrobial agent (c), the one or more than one additional antimicrobial agent (c) preferably constitutes no greater than 50 wt % of the total weight of the antimicrobial composition, typically a minority (<50 wt %) of the antimicrobial composition or a minority by weight relative to the combined amount of the antimicrobial agent (a) and the pelargonic acid and/or salt thereof (b), such as no greater than 40 wt % or no greater than 30 wt %, more preferably no greater than 25 wt %, no greater than 20 wt %, even more preferably no greater than 15 wt %, no greater than 10 wt %, no greater than 5 wt %, or no greater than 3 wt %, based on the total weight of the antimicrobial composition.

The antimicrobial composition may preferably contain from about 70 to 100 wt %, from about 75 to 100 wt %, from about 80 to 100 wt %, from about 85 to 100 wt %, or from about 90 to 100 wt %, or preferably from about 70 wt %, more preferably from about 75 wt %, from about 80 wt % or from about 85 wt % to about 99 wt %, to about 97 wt %, to about 95 wt % or to about 92 wt %, of the antimicrobial agent (a), the pelargonic acid and/or salt thereof (b) and the one or more than one additional antimicrobial agent (c) combined, based on the total weight of the antimicrobial composition. The antimicrobial agent (a), the pelargonic acid and/or salt thereof (b) and the one or more than one additional antimicrobial agent (c) may constitute all or substantially all of the ingredients in the antimicrobial composition. In many embodiments, the antimicrobial composition consists essentially of, or in further embodiments consists of, the antimicrobial agent (a), the pelargonic acid and/or salt thereof (b) and the one or more than one additional antimicrobial agent (c).

Typically, the ratio by weight of the combined amount of (a) and (b) to the amount of the one or more than one additional antimicrobial agent (c) ranges from about 1:1 to about 150:1, such as from about 1.1:1, from about 1.2:1, from about 1.5:1, from about 2:1, from about 3:1, from about 4:1, preferably from about 5:1, from about 9:1, more preferably from about 12:1, from about 16:1, even more preferably from about 20:1 to about 150:1, preferably to about 100:1, more preferably to about 75:1 or to about 50:1.

The antimicrobial composition of the present disclosure may, but need not, comprise added solvent (d). One advantage of the presently disclosed combination of the antimicrobial agent (a) and the pelargonic acid and/or salt thereof (b) is that the materials can be combined without requiring added solvent. Thus, in many embodiments, the antimicrobial composition is free or substantially free of solvent. Preferably, the amount of the pelargonic acid and/or salt thereof (b) does not exceed a ratio of the antimicrobial agent (a) to the pelargonic acid and/or salt thereof (b) of 1:2.5, such as not exceeding 1:2, not exceeding 1:1.5 or not exceeding 1:1.25.

If present, the one or more than one solvent (d) typically constitutes from about 10 wt %, from about 20 wt % or from about 30 wt % to about 90 wt %, to about 85 wt %, to about 80 wt %, to about 70 wt %, to about 60 wt % or to about 50 wt %, based on the total weight of the antimicrobial composition. Solvents may be chosen according to their compatibility or suitability for a desired end application or end product formulation, e.g., in a personal care formulation (e.g., a cosmetically acceptable solvent) or a home care formulation. Suitable solvents include, but are not limited to, water, C₁-C₄ alcohols, C₁-C₄ diols (e.g., glycols), glycerin and the like.

The antimicrobial composition may, but need not, contain one or more excipients (e). Excipients may similarly be chosen according to their compatibility or suitability for a desired end application or end product formulation, e.g., in a personal care formulation (e.g., a cosmetically acceptable excipient) or a home care formulation. Examples of excipients include, but are not limited to, a dispersant, solubilizer, buffer, and/or stabilizer. As similarly described above with respect to a solvent, one advantage of the presently disclosed combination of the antimicrobial agent (a) and the pelargonic acid and/or salt thereof (b) is that the materials can be combined without requiring a solubilizing or miscibility agent. Thus, in many embodiments, the antimicrobial composition is free or substantially free of solubilizing or miscibility agents. Preferably, the amount of the pelargonic acid and/or salt thereof (b) does not exceed a ratio of the antimicrobial agent (a) to the pelargonic acid and/or salt thereof (b) of 1:2.5, such as not exceeding 1:2, not exceeding 1:1.5 or not exceeding 1:1.25.

The antimicrobial composition may comprise one or more excipients (e), for example, from 0 to about 25 wt %, such as from about 0.001 wt %, from about 0.01 wt %, from about 0.05 wt % or from about 0.1 wt % to about 20 wt %, to about 15 wt %, to about 10 wt %, to about 5 wt %, to about 3 wt %, or to about 1 wt %, based on the total weight of the antimicrobial composition. For example, the component (e) may range from 0 to about 20 wt %, from 0 to about 15 wt %, from 0 to about 10 wt %, from 0 to about 5 wt %, or from 0 to about 3 wt %, or from about 0.01 wt % to about 25 wt %, to about 20 wt %, to about 15 wt %, to about 10 wt % or to about 5 wt %, from about 0.1 wt % to about 25 wt %, to about 20 wt %, to about 15 wt %, to about 10 wt % or to about 5 wt %, from about 0.01 to about 3 wt %, from about 0.1 to about 3 wt %, from about 0.01 to about 1 wt %, or from about 0.1 to about 1 wt %.

In accordance with the present disclosure, it was found that the stability over time of the presently disclosed antimicrobial composition can be significantly enhanced via the inclusion of an alkaline stabilizer in the composition. Therefore, preferably, the at least one excipient (e) includes at least one alkaline stabilizer. The alkaline stabilizer comprises a base preferably chosen from a metal (preferably an alkali metal or alkaline earth metal) hydroxide, oxide, carbonate, bicarbonate, phosphate, or hydride, an amine base or a combination thereof, more preferably a metal hydroxide. The metals are preferably chosen from sodium, potassium, magnesium and calcium. In many preferred embodiments, the alkaline stabilizer comprises sodium hydroxide, potassium hydroxide or a combination thereof. In particular, the alkaline stabilizer may be chosen to ensure compatibility or suitability for a desired end application or end product formulation, e.g., in a personal care formulation (e.g., a cosmetically acceptable alkaline stabilizer) or a home care formulation. The base of the alkaline stabilizer may be present, for example, in an amount of from about 0.1 wt %, from about 0.3 wt %, preferably from about 0.5 wt %, more preferably from about 1 wt % or from about 3 wt % to about 10 wt %, preferably to about 8 wt % or to about 6 wt %, based on the total weight of the antimicrobial composition. Typically, the alkaline stabilizer is in the form of an alkaline solution, preferably an alkaline aqueous solution. For example, the alkaline solution, such as a metal hydroxide solution (e.g., where the metal hydroxide comprises potassium hydroxide, sodium hydroxide or a combination thereof), may have a strength of the base component in the solution of from about 1% to about 90%, to about 80%, to about 70% or to about 60% (w/w), preferably from about 10%, from about 20% or from about 30% to about 70%, to about 60% or to about 50% (w/w). Higher strength alkaline solutions may also be used. Preferably, the alkaline stabilizer has a pH ranging from about 9.0, more preferably from about 10.0 to about 14.0, such as from about 11.0 or from about 12.0 to about 14.0 or to about 13.0. With the inclusion of the alkaline stabilizer, the antimicrobial composition preferably has a pH ranging from about 5.0 to about 7.5, more preferably from about 5.5 or from about 6.0 to about 7.5, to about 7.0 or to about 6.5. If included, the alkaline stabilizer typically constitutes from about 0.2 wt %, from about 0.5 wt %, or from about 1 wt % to about 25 wt %, preferably from about 1 wt %, more preferably from about 3 wt % or from about 5 wt % to about 20 wt % or to about 15 wt %, such as from about 5 wt % or from about 8 wt % to about 15 wt % or to about 12 wt %, based on the total weight of the antimicrobial composition. As shown in the Examples section, the stabilized antimicrobial composition preferably exhibits≤5 wt % loss of the antimicrobial agent (a) and the pelargonic acid (and/or salt thereof) (b) combined when stored at 55° C. for 2 weeks. As further shown in the Examples section, the stabilized antimicrobial composition preferably exhibits≤15 wt % loss, more preferably ≤10 wt % loss, even more preferably ≤5 wt % loss, of the antimicrobial agent (a) and the pelargonic acid (and/or salt thereof) (b) combined when stored at 55° C. for 5 weeks.

In general, the antimicrobial composition may comprise a combined amount of the antimicrobial agent (a) and the pelargonic acid and/or salt thereof (b) of from about 10 to 100 wt %, such as from about 15 wt % or from about 20 wt %, from about 30 wt %, from about 40 wt %, from about 50 wt % or from about 60 wt % to about 100 wt %, to about 99 wt %, to about 97 wt %, to about 95 wt %, to about 92 wt %, to about 85 wt %, to about 80 wt %, or to about 70 wt %, based on the total weight of the antimicrobial composition; optionally one or more than one additional antimicrobial agent (c), typically at a weight ratio of the combined amount of (a) and (b) to the amount of (c) as described herein; optionally one or more solvents (d) in an amount from 0 to about 90 wt %, such as from about 10 wt %, from about 20 wt % or from about 30 wt % to about 90 wt %, to about 85 wt %, to about 80 wt %, to about 70 wt %, to about 60 wt %, to about 50 wt %, or to about 40 wt %, based on the total weight of the antimicrobial composition; and optionally one or more excipients (e) in an amount from about 0 to about 25 wt %, to about 20 wt %, to about 15 wt %, or to about 10 wt %, such as from about 0.001 wt %, from about 0.01 wt %, from about 0.05 wt % or from about 0.1 wt % to about 20 wt %, to about 15 wt %, to about 10 wt %, to about 5 wt %, to about 3 wt %, or to about 1 wt %, or, particularly in the case of the alkaline stabilizer excipient, from about 0.2, from about 0.5, or from about 1 wt % to about 25 wt %, preferably from about 3 wt %, more preferably from about 5 wt % to about 20 wt %, such as from about 5 wt % or from about 8 wt % to about 15 wt % or to about 12 wt %, based on the total weight of the antimicrobial composition.

In many embodiments, the antimicrobial composition consists essentially of, or in further embodiments consists of, the antimicrobial agent (a), the pelargonic acid and/or salt thereof (b), optionally one or more than one additional antimicrobial agent (c), optionally one or more than one solvent (d) and optionally one or more than one excipient (e). In many embodiments, the antimicrobial composition consists essentially of, or in further embodiments consists of, the antimicrobial agent (a), the pelargonic acid and/or salt thereof (b), one or more than one additional antimicrobial agent (c), optionally one or more than one solvent (d) and optionally one or more than one excipient (e). In further embodiments, a stabilized antimicrobial composition consists essentially of, or in further embodiments consists of, the antimicrobial agent (a), the pelargonic acid and/or salt thereof (b), one or more than one excipient (e) comprising an alkaline stabilizer, optionally one or more than one additional antimicrobial agent (c), and optionally one or more than one solvent (d). In additional embodiments, a stabilized antimicrobial composition consists essentially of, or in further embodiments consists of, the antimicrobial agent (a), the pelargonic acid and/or salt thereof (b), one or more than one additional antimicrobial agent (c), one or more than one excipient (e) comprising an alkaline stabilizer, and optionally one or more than one solvent (d).

The one or more than one additional antimicrobial agent (c) of the present disclosure may be chosen based on suitability for the intended application, such as in personal care or home care formulations, and are preferably suitably selected to be sufficiently soluble with the combination of the antimicrobial agent (a) and the pelargonic acid (b) either in the absence of any added solvent or in a chosen solvent for the antimicrobial composition.

Examples of the additional antimicrobial agents (c) include, but are not limited to, alkylhydroxamic acids and/or salts thereof, aromatic aldehydes (e.g., cinnamic aldehyde), aromatic alcohols (e.g., cinnamic alcohol, dichlorobenzyl alcohol, phenolic compounds, such as hydroxyacetophenone), diols (e.g., glycols, such as 1,2-propanediol, 1,2-butanediol, 1,2-pentanediol, 1,2-hexanediol, 1,2-heptanediol, and 1,2-octanediol (i.e., caprylyl glycol), bronopol), glyceryl ethers (e.g., ethylhexylglycerin, chlorphenesin), isothiazolinones (e.g., methylisothiazolinone, chloromethylisothiazolinone, benzisothiazolinone), fatty acids and/or salts thereof, other than pelargonic acid and salts thereof, (e.g., fatty acids and salts thereof having up to 24 carbon atoms, such as up to 20, up to 16 or up to 12 carbons atoms, particularly, e.g., C₆-C₈ and C₁₀-C₁₂ fatty acids and salts thereof), pyrithiones (e.g., zinc pyrithione, sodium pyrithione) and combinations thereof. It shall be understood that the scope of the one or more than one additional antimicrobial agent (c) does not include the antimicrobial agent (a) or the pelargonic acid and/or salt thereof (b).

Examples of suitable alkylhydroxamic acids and salts thereof include those having at least one alkyl group of a chain length of from about 2 to about 22 carbon atoms, which may be branched or linear in structure, substituted (e.g., alkoxy and the like) or unsubstituted, and saturated or unsaturated (e.g., alkenyl, alkynyl, alkenoxy, alkynoxy and the like). For example, the carbon chains may be interrupted by one or more oxygen atoms and/or may contain one or more side- and/or terminal-hydroxy group substituents. Other functional groups, particularly groups compatible with or suggested for use in personal care or home care formulations, may be used. Preferably, the alkylhydroxamic acids and/or salts thereof contain alkyl groups having a chain length, often linear, of about 6 to about 12 carbon atoms. Examples of suitable alkylhydroxamic acids and salts thereof include hexanohydroxamic acid, caprylhydroxamic acid, decanohydroxamic acid, laurohydroxamic acid and salts and combinations thereof, preferably caprylhydroxamic acid and/or a salt thereof. More than one alkylhydroxamic acid and/or salt thereof may be used.

The one or more than one additional antimicrobial agent (c) preferably comprises an alkylhydroxamic acid and/or salt thereof (more preferably, hexanohydroxamic acid, caprylhydroxamic acid, decanohydroxamic acid, laurohydroxamic acid and/or salt thereof or combination thereof, particularly caprylhydroxamic acid and/or salt thereof), hydroxyacetophenone, dichlorobenzyl alcohol, cinnamic aldehyde, cinnamic alcohol, 1,2-propanediol, 1,2-butanediol, 1,2-pentanediol, 1,2-hexanediol, 1,2-heptanediol, 1,2-octanediol (i.e., caprylyl glycol), bronopol, ethylhexylglycerin, chlorphenesin, methylisothiazolinone, chloromethylisothiazolinone, benzisothiazolinone, a fatty acid and/or salt thereof (e.g., a C₆ to C₈ or C₁₀ to C₁₂ fatty acid and/or salt thereof, preferably, a C₆, C₇, C₈, or C₁₀, particularly C₈ or C₁₀, fatty acid and/or salt thereof or combination thereof), zinc pyrithione, sodium pyrithione or a combination thereof.

More preferably, the one or more than one additional antimicrobial agent (c) comprises an alkylhydroxamic acid and/or salt thereof (such as any exemplary alkylhydroxamic acid and/or salt thereof as described herein, particularly caprylhydroxamic acid and/or a salt thereof), cinnamic aldehyde, a C₆ to C₈ or C₁₀ to C₁₂ fatty acid and/or salt thereof, zinc pyrithione, sodium pyrithione or a combination thereof.

In particularly preferred embodiments, the one or more than one additional antimicrobial agent (c) comprises or is an alkylhydroxamic acid and/or salt thereof, such as any exemplary alkylhydroxamic acid and/or salt thereof as described herein, preferably caprylhydroxamic acid and/or a salt thereof.

In a further aspect, the antimicrobial composition, such as any embodiment of the antimicrobial composition disclosed herein, is free or substantially free of any dicarboxylic acid or salt thereof. In another aspect, the antimicrobial composition, such as any embodiment of the antimicrobial composition disclosed herein, is free or substantially free of glutaric acid, adipic acid, pimelic acid or salts thereof. In a further aspect, the antimicrobial composition, such as any embodiment of the antimicrobial composition disclosed herein, is free or substantially free of any aromatic carboxylic acid or salt thereof. In another aspect, the antimicrobial composition, such as any embodiment of the antimicrobial composition disclosed herein, is free or substantially free of benzoic acid, p-hydroxybenzoic acid, p-methoxybenzoic acid, salicylic acid, cinnamic acid, gentianic acid, caffeic acid or salts thereof.

The present disclosure is not limited to any particular technique for preparing the antimicrobial composition and such composition may be prepared in any suitable manner for combining or mixing the components described herein.

The present disclosure provides for the use of an antimicrobial effective amount of the presently disclosed antimicrobial composition in a wide range of product formulations and applications, such as for use in personal care (e.g., cosmetic), home care, health care (such as pharmaceutical (i.e., having at least one active pharmaceutical ingredient) or other health care products) or industrial product formulations, particularly formulations for use as a personal care or home care product. Examples include, without limitation, household products and cleaners, fabric detergents, dish detergents, cleansers, soaps, bubble baths, disinfectants, deodorizers, pharmaceutical products, cosmetics, hygiene compositions, infant care products, antimicrobial soaps, hand sanitizers, deodorants, antiperspirants, anti-microbial coatings, wound care compositions, dermatological compositions, skin conditioners, skin moisturizers, anti-wrinkle formulations, sunscreens, tanning lotions, hair products, shampoos, shower gels, bubble baths, conditioners, shaving creams, etc. Depending on the application, the product formulation may contain numerous and different compatible ingredients. In many embodiments, the product formulation comprises an antimicrobial effective amount of the antimicrobial composition of the present disclosure and one or more additional ingredients suitable for use in a personal care (e.g., cosmetic), home care, health care, or industrial product formulation, particularly a personal care or home care formulation.

In one aspect, an aqueous product formulation comprises an antimicrobial effective amount of the antimicrobial composition of the present disclosure. Preferably, the aqueous product formulation is a personal care (e.g., cosmetic), home care, health care, or industrial product formulation, particularly a personal care or home care product formulation.

The aqueous product formulation typically has a pH ranging from about 3.0, from about 4.0, from about 5.0, from about 5.5, from about 6.0 or from about 6.5 to about 12 or to about 11, such as from about 3.0 to about 10, from about 3.0 to about 8.5, from about 4.0 to about 8.5, from about 3.0 to about 8.0, from about 4.0 to about 8.0, from about 3.0 to about 7.5, from about 4.0 to about 7.5 or from about 5.0 to about 7.5. As provided herein, pH values or ranges thereof refer to pH at room temperature (20-25° C.), unless otherwise specified. The selected pH will vary depending, for example, on the particular end use application (e.g., home care or personal care applications).

As demonstrated in the present disclosure, it was unexpectedly found that the enhanced antimicrobial activity of the antimicrobial agent (a) when combined with the pelargonic acid and/or salt thereof (b) is achieved in product formulations having a pH above (even well above) the pKa of pelargonic acid (pKa of 4.95 at 25° C.). Accordingly, in many embodiments, the aqueous product formulation has a pH ranging from about 5.0, from about 5.1, from about 5.2, from about 5.3, from about 5.4, from about 5.5, from about 5.6, from about 5.7, from about 5.8, from about 5.9, from about 6.0, from about 6.1, from about 6.2, from about 6.3, from about 6.4 or from about 6.5 to about 12, to about 11, to about 10, to about 9.5, to about 9.0, to about 8.5, to about 8.4, to about 8.3, to about 8.2, to about 8.1, to about 8.0, to about 7.9, to about 7.8, to about 7.7, to about 7.6 or to about 7.5. The pH may range, for example, from about 5.0, from about 5.5, from about 6.0 or from about 6.5 to about 12, to about 11, to about 10, to about 9.0, to about 8.5, to about 8.0 or to about 7.5. In many embodiments, the pH is higher than 5.0, higher than 5.5, higher than 6.0, or higher than 6.5, preferably up to about 11, up to about 10, up to about 9.0, up to about 8.5, up to about 8.0 or up to about 7.5.

The pH of the aqueous product formulation is more preferably at least 6.0, even more preferably higher than 6.0, such as a pH of at least 6.1, at least 6.3, most preferably at least 6.5, at least 6.7, at least 6.9, at least 7.0, at least 7.1, at least 7.2, at least 7.3, at least 7.4, or at least 7.5. The pH of the aqueous product formulation may be up to about 12, preferably up to about 11 or up to about 10.

As a particular example, the antimicrobial composition of the present disclosure can be incorporated into any number and variety of different personal care products, such as cosmetic products. Accordingly, a personal care formulation is provided comprising an antimicrobial effective amount of a combination of the antimicrobial agent (a), the pelargonic acid and/or salt thereof (b) and optionally the one or more than one additional antimicrobial agent (c), and the formulation further comprises one or more additional ingredients suitable for use in a personal care formulation. The personal care formulation may be, for example, any formulation for skin care, hair care, cosmetics, personal cleaning, hygiene, sun protection and other personal care applications. Examples of such products include, without limitation, skin toners, skin cleansers, night creams, skin creams, shaving creams, skin lotions, makeup, mascara, lipstick, blush, gloss, eye-liner, makeup removers, sunscreens, lip balms, fragrances, massage oils, shampoos, conditioners, hair styling gels, hair reparatives, hair tonics, hair fixatives, hair mousses, bath and shower gels, liquid soaps, moisturizing sprays, bath additives, ophthalmic preparations, foaming soaps and body washes, liquids for any personal care wet wipe application, etc.

Depending on the application, the personal care product formulation may contain numerous and different compatible ingredients. A personal care formulation may contain, for example, any of solvents, surfactants, emulsifiers, chelating agents, oxidizing agents, colorants, rheology modifiers, conditioners, emollients, skin care ingredients, moisturizers, thickeners, humectants, fillers, antioxidants, active ingredients, such as dermatologically active ingredients typically suited for topical application, fragrances, etc.

The personal care formulation preferably has a pH ranging from about 3.0 or from about 4.0 to about 8.0, such as from about 3.0 or from about 4.0 to about 7.5. The pH may range, for example, from about 5.0, from about 5.5, from about 6.0 or from about 6.5 to about 8.0, to about 7.5 or to about 7.0.

The pH of the personal care product formulation is more preferably at least 6.0, even more preferably higher than 6.0, such as a pH of at least 6.1, at least 6.3, most preferably at least 6.5, at least 6.7, at least 6.9, at least 7.0, at least 7.1, at least 7.2, at least 7.3, at least 7.4, or at least 7.5. The pH of the personal care formulation is preferably up to about 8.0.

As a further example, the antimicrobial composition of the present disclosure can be incorporated into any number and variety of different home care products, such as fabric care products and cleaning products. Accordingly, a home care formulation is provided comprising an antimicrobial effective amount of a combination of the antimicrobial agent (a), the pelargonic acid and/or salt thereof (b) and optionally the one or more than one additional antimicrobial agent (c), and the formulation further comprises one or more additional ingredients suitable for use in a home care formulation. Examples include dish soaps, laundry detergents, cleaning wipes, cleaning formulations and other home care applications.

Depending on the application, the home care product formulation may contain, for example, any of cleaning agents, detergents, emulsifiers, surfactants, thickeners, gelling agents, bleaches, whiteners, deodorizers, enzymes, stabilizers, fragrances, soil-release agents, anti-shrinking agents, anti-wrinkle agents, anti-spotting agents, antioxidants, UV absorbing compounds, anti-corrosion agents, anti-static agents, ironing aids, odor-preventing compounds, etc.

The home care formulation preferably has a pH ranging from about 3.0 to about 11, such as from about 3.0 or from about 4.0 to about 10. The pH may range, for example, from about 5.0, from about 5.5, from about 6.0, from about 6.5 or from about 7.0 to about 11, to about 10, to about 9.0, to about 8.5, to about 8.0 or to about 7.5. The pH of the home care product formulation is more preferably at least 6.0, even more preferably higher than 6.0, such as a pH of at least 6.1, at least 6.3, most preferably at least 6.5, at least 6.7, at least 6.9, at least 7.0, at least 7.1, at least 7.2, at least 7.3, at least 7.4, or at least 7.5. The pH of the home care formulation preferably may be up to about 12 or up to about 11.

Examples of the foregoing ingredients and other agents used in personal care and home care product compositions are known in the art.

The antimicrobial composition of the present disclosure may be included in product formulations of a variety of different forms, for example, liquids, pastes, serums, hydrogels, creams, emulsions, lotions, gels, oils, wipes, ointments, semi-solid compositions, foams and aerosol sprays. The antimicrobial composition may be used in a leave-on or rinse-off product formulation. The antimicrobial composition can be used in hair care products, such as shampoos, hair conditioners, hair dyes, hair tonic, hair gel, hair dressings, hair grooming aids and other hair care preparations; shaving applications such as shaving cream, aftershave lotions, and other shaving applications; personal cleaners for the body and hands, such as liquid bath soaps and detergents; fragrance preparations, such as perfumes, after bath splashes, and other similar fragrant preparations, skin care products, such as moisturizers, creams, and lotions and other similar skin care products, make-up products, such as mascara, base foundations and the like; make-up removal products, sun care products, indoor tanning products and other similar personal care products. The antimicrobial composition may be incorporated in formulations used to saturate wipes used for personal cleaning and hygiene, for example baby wipes, wet toilet wipes, make-up removal wipes and exfoliating wipes the like, or wet wipe formulations for home care.

An optimal ratio of the antimicrobial agent (a) to the pelargonic acid and/or salt thereof (b), an optimal ratio of the amount of components (a) and (b) combined to the amount of the one or more additional antimicrobial agents (c) (if used), and an optimal antimicrobial effective amount of the antimicrobial composition may vary depending, for example, on the particular components selected, the end use application, the desired level of antimicrobial activity, the final properties of the product, and the like. As known in the art, an antimicrobial effect may be demonstrated by various suitable antimicrobial effectiveness tests. Preferably, when added to a product formulation, the antimicrobial composition provides an antimicrobial effect according to the challenge test procedures in the below Examples achieving a passing growth rating according to the criteria associated with Table 8 and the associated description herein.

In general, the antimicrobial effective amount of the antimicrobial composition provides from about 0.05 wt %, preferably from about 0.1 wt %, more preferably from about 0.2 wt %, from about 0.3 wt %, from about 0.4 wt % or from about 0.5 wt % to about 5 wt %, to about 4 wt %, preferably to about 3 wt %, to about 2.5 wt %, more preferably to about 2 wt %, to about 1.5 wt % or to about 1 wt % of the antimicrobial agent (a) and the pelargonic acid and/or salt thereof (b) combined, based on the total weight of the formulation, and the formulation may optionally comprise one or more than one additional antimicrobial agent (c), preferably in the ratio by weight of the combined amount of (a) and (b) to the amount of (c) as described herein. If the optional component (c) is included (preferably in the ratio by weight of the combined amount of (a) and (b) to the amount of (c) as described herein), the antimicrobial effective amount may provide, for example, from about 0.2 wt %, from about 0.3 wt %, from about 0.4 wt % or from about 0.5 wt % to about 6 wt %, to about 5 wt %, to about 4 wt %, preferably to about 3 wt %, more preferably to about 2 wt % or to about 1 wt % of components (a), (b) and (c) combined, based on the total weight of the formulation. Typically, the antimicrobial agent (a) and the pelargonic acid and/or salt thereof (b) each is present in a product formulation in an amount not exceeding 3 wt %, preferably not exceeding 2 wt %, not exceeding 1.5 wt % or not exceeding 1 wt %, based on the total weight of the formulation. Often, each is present below 1 wt %. Preferably, the combined amount of the antimicrobial agent (a), the pelargonic acid and/or salt thereof (b) and, if used, the optional one or more than one additional antimicrobial agent (c) ranges from about 2 wt % or less (e.g., from about 0.1 wt %, from about 0.2 wt % or from about 0.3 wt % to about 2 wt %), from about 1.5 wt % or less (e.g., from about 0.1 wt %, from about 0.2 wt % or from about 0.3 wt % to about 1.5 wt %), from about 1.2 wt % or less (e.g., from about 0.1 wt %, from about 0.2 wt % or from about 0.3 wt % to about 1.2 wt %) or from about 1 wt % or less (e.g., from about 0.1 wt %, from about 0.2 wt % or from about 0.3 wt % to about 1 wt %), based on the total weight of the formulation. In many embodiments, the formulation comprises no greater than 1.5 wt %, preferably no greater than 1 wt %, no greater than 0.9 wt %, no greater than 0.8 wt %, no greater than 0.7 wt %, no greater than 0.6 wt %, more preferably no greater than 0.5 wt %, no greater than 0.4 wt % or no greater than 0.3 wt % of the antimicrobial agent (a). Preferably, the formulation comprises no greater than 0.5 wt %, no greater than 0.4 wt %, no greater than 0.3 wt %, more preferably no greater than 0.2 wt %, even more preferably no greater than 0.1 wt % or no greater than 0.05 wt % of the one or more than one additional antimicrobial agent (c).

The product formulation may comprise, for example, the antimicrobial agent (a) and the pelargonic acid and/or salt thereof (b) in a combined antimicrobial effective amount of from about 0.2 wt %, from about 0.3 wt %, from about 0.4 wt % or from about 0.5 wt % to about 5 wt %, preferably to about 3 wt %, more preferably to about 2 wt %, to about 1.5 wt % or to about 1 wt %, based on the total weight of the formulation, and further comprise the one or more than one additional antimicrobial agent (c), wherein the ratio by weight of the combined amount of (a) and (b) to the amount of (c) ranges from about 1:1 to about 150:1, such as from about 1.1:1, from about 1.2:1, from about 1.5:1, from about 2:1, from about 3:1, from about 4:1, preferably from about 5:1, from about 9:1, more preferably from about 12:1, from about 16:1, particularly preferably from about 20:1 to about 150:1, preferably to about 100:1, more preferably to about 75:1 or to about 50:1. Preferably, the one or more than one additional antimicrobial agent (c) comprises or is at least one alkylhydroxamic acid as described herein and/or a salt thereof, more preferably caprylhydroxamic acid and/or a salt thereof.

The product formulation may comprise, for example, the antimicrobial agent (a) and the pelargonic acid and/or salt thereof (b) in a combined antimicrobial effective amount of from about 0.2 wt %, from about 0.3 wt %, from about 0.4 wt % or from about 0.5 wt % to about 5 wt %, preferably to about 3 wt %, more preferably to about 2 wt %, to about 1.5 wt % or to about 1 wt %, and further comprise the one or more than one additional antimicrobial agent (c) in an amount less than or equal to about 0.2 wt %, such as no greater than 0.15 wt %, no greater than 0.1 wt % or no greater than 0.05 wt %, each based on the total weight of the formulation. Preferably, the one or more than one additional antimicrobial agent (c) comprises or is at least one alkylhydroxamic acid as described herein and/or a salt thereof, more preferably caprylhydroxamic acid and/or a salt thereof.

The present disclosure is not limited to any particular technique for preparing a product formulation, and the antimicrobial composition of the present disclosure may be added to a product formulation in any suitable manner. For example, the antimicrobial agent (a), the pelargonic acid and/or salt thereof (b), the optional one or more than one additional antimicrobial agent (c) and the other ingredients as described herein can be applied to a product formulation as a mixture or applied individually to the formulation at the same time or in any order.

The present disclosure further includes a method of controlling or inhibiting the growth of microorganisms in a product formulation, such as any formulation described herein. Also disclosed is a method of preserving a product formulation, such as any formulation described herein. The methods comprise adding to the formulation an antimicrobial effective amount of the antimicrobial composition as described herein.

In another aspect, the present disclosure includes a method of enhancing the antimicrobial efficacy of an antimicrobial agent selected from the group consisting of phenoxyethanol, benzyl alcohol and combinations thereof, comprising combining or mixing pelargonic acid and/or a salt thereof with the antimicrobial agent in a ratio by weight as described herein. The method may include combining or mixing the pelargonic acid and/or salt thereof with the antimicrobial agent in a product formulation, such as any formulation described herein.

Also provided is the use of pelargonic acid and/or a salt thereof to enhance the antimicrobial efficacy of an antimicrobial agent selected from the group consisting of phenoxyethanol, benzyl alcohol and combinations thereof, wherein the ratio by weight of the antimicrobial agent to the pelargonic acid and/or salt thereof is as described herein.

Additionally provided is a method of reducing the minimum amount of an antimicrobial agent needed for preservation activity in a product formulation, such as any formulation described herein, the method comprising combining or mixing pelargonic acid and/or a salt thereof with the antimicrobial agent in the product formulation, wherein the antimicrobial agent is selected from the group consisting of phenoxyethanol, benzyl alcohol and combinations thereof, and the ratio by weight of the antimicrobial agent to the pelargonic acid and/or salt thereof is as described herein.

The present disclosure further includes, without limitation, the following exemplary embodiments.
Embodiment 1: An antimicrobial composition comprising (a) an antimicrobial agent selected from the group consisting of phenoxyethanol, benzyl alcohol and combinations thereof, and (b) pelargonic acid and/or a salt thereof, wherein the ratio by weight of the antimicrobial agent (a) to the pelargonic acid and/or salt thereof (b) is from about 40:1 to about 1:5, preferably from about 40:1 or from about 20:1 to about 1:2.5.
Embodiment 2: The antimicrobial composition of Embodiment 1, wherein the antimicrobial agent (a) is phenoxyethanol.
Embodiment 3: The antimicrobial composition of Embodiment 1, wherein the antimicrobial agent (a) is benzyl alcohol.
Embodiment 4: The antimicrobial composition of Embodiment 1, wherein the antimicrobial agent is a combination of phenoxyethanol and benzyl alcohol.
Embodiment 5: The antimicrobial composition of any one of Embodiments 1-4, wherein the component (b) is pelargonic acid.
Embodiment 6: The antimicrobial composition of any one of Embodiments 1-5, wherein the antimicrobial composition contains greater than or equal to about 50 wt % of the antimicrobial agent (a) and the pelargonic acid and/or salt thereof (b) combined, based on the total weight of active ingredients in the antimicrobial composition, preferably from about 60 to 100 wt %, from about 70 to 100 wt %, from about 75 to 100 wt %, from about 80 to 100 wt %, from about 85 to 100 wt %, from about 90 to 100 wt %, or from about 95 or from about 97 to 100 wt % of the antimicrobial agent (a) and the pelargonic acid and/or salt thereof (b) combined, based on the total weight of active ingredients in the antimicrobial composition.
Embodiment 7: The antimicrobial composition of any one of Embodiments 1-5, wherein the antimicrobial composition comprises active ingredients consisting essentially of, or in further embodiments consisting of, the antimicrobial agent (a) and the pelargonic acid and/or salt thereof (b).
Embodiment 8: The antimicrobial composition of any one of Embodiments 1-5, wherein the antimicrobial composition contains greater than or equal to about 50 wt % of the antimicrobial agent (a) and the pelargonic acid and/or salt thereof (b) combined, based on the total weight of the antimicrobial composition, preferably from about 60 to 100 wt %, from about 70 to 100 wt %, from about 75 to 100 wt %, from about 80 to 100 wt %, from about 85 to 100 wt %, or from about 90 to 100 wt %, or from about 60 wt %, preferably from about 70 wt %, more preferably from about 75 wt %, from about 80 wt % or from about 85 wt % to about 99 wt %, to about 97 wt %, to about 95 wt % or to about 92 wt % of the antimicrobial agent (a) and the pelargonic acid and/or salt thereof (b) combined, based on the total weight of the composition.
Embodiment 9: The antimicrobial composition of any one of Embodiments 1-5, wherein the composition consists essentially of, or in further embodiments consists of, the antimicrobial agent (a), the pelargonic acid and/or salt thereof (b), optionally one or more than one solvent (d) and optionally one or more than one excipient (e).
Embodiment 10: The antimicrobial composition of Embodiment 6, wherein the antimicrobial composition further comprises one or more than one additional antimicrobial agent (c) in an amount no greater than 50 wt %, preferably no greater than 40 wt %, no greater than 25 wt %, no greater than 20 wt %, no greater than 15 wt %, no greater than 10 wt % or no greater than 5 wt %, based on the total weight of active ingredients in the antimicrobial composition, preferably wherein the one or more than one additional antimicrobial agent (c) comprises an alkylhydroxamic acid and/or salt thereof, an aromatic aldehyde (e.g., cinnamic aldehyde), an aromatic alcohol (e.g., cinnamic alcohol, dichlorobenzyl alcohol, a phenolic compound, such as hydroxyacetophenone), a diol (e.g., a glycol, such as 1,2-propanediol, 1,2-butanediol, 1,2-pentanediol, 1,2-hexanediol, 1,2-heptanediol, or 1,2-octanediol (i.e., caprylyl glycol), bronopol), a glyceryl ether (e.g., ethylhexylglycerin, chlorphenesin), an isothiazolinone (e.g., methylisothiazolinone, chloromethylisothiazolinone, benzisothiazolinone), a fatty acid and/or salt thereof (e.g., a fatty acid and/or salt thereof having up to 24 carbon atoms, such as up to 20, up to 16 or up to 12 carbons atoms, particularly, e.g., a C₆-C₈ or C₁₀-C₁₂ fatty acid and/or salt thereof), a pyrithione (e.g., zinc pyrithione, sodium pyrithione) or a combination thereof.
Embodiment 11: The antimicrobial composition of Embodiment 8, wherein the antimicrobial composition further comprises one or more than one additional antimicrobial agent (c) in an amount no greater than 50 wt %, preferably no greater than 40 wt %, no greater than 25 wt %, no greater than 20 wt %, no greater than 15 wt %, no greater than 10 wt % or no greater than 5 wt %, based on the total weight of the antimicrobial composition, preferably wherein the one or more than one additional antimicrobial agent (c) is as described in Embodiment 10 above.
Embodiment 12: The antimicrobial composition of any one of Embodiments 1-5, wherein the antimicrobial composition comprises active ingredients consisting essentially of, or in further embodiments consisting of, the antimicrobial agent (a), the pelargonic acid and/or salt thereof (b) and one or more than one additional antimicrobial agent (c), wherein the one or more than one additional antimicrobial agent (c) is present in a minority amount relative to the combined amount of the antimicrobial agent (a) and the pelargonic acid and/or salt thereof (b), preferably wherein the one or more than one additional antimicrobial agent (c) is as described in Embodiment 10 above.
Embodiment 13: The antimicrobial composition of any one of Embodiments 10-12, wherein the ratio by weight of the combined amount of (a) and (b) to the amount of the one or more than one additional antimicrobial agent (c) ranges from about 1.5:1 to about 150:1, such as from about 2:1, from about 3:1, from about 4:1, preferably from about 5:1, from about 9:1, more preferably from about 12:1, from about 16:1, particularly preferably from about 20:1 to about 150:1, preferably to about 100:1, more preferably to about 75:1 or to about 50:1.
Embodiment 14: The antimicrobial composition of Embodiment 6, wherein the antimicrobial composition comprises active ingredients consisting essentially of, or in further embodiments consisting of, the antimicrobial agent (a), the pelargonic acid and/or salt thereof (b), and optionally one or more than one additional antimicrobial agent (c), wherein from about 60 to 100 wt %, preferably from about 70 to 100 wt %, from about 75 to 100 wt %, from about 80 to 100 wt %, from about 85 to 100 wt %, from about 90 to 100 wt %, or from about 95 or from about 97 to 100 wt %, based on the total weight of active ingredients in the antimicrobial composition, is the antimicrobial agent (a) and the pelargonic acid and/or salt thereof (b) combined, preferably wherein the one or more than one additional antimicrobial agent (c) is as described in Embodiment 10 above.
Embodiment 15: The antimicrobial composition of any one of Embodiments 1-5, wherein the antimicrobial composition consists essentially of, or in further embodiments consists of, the antimicrobial agent (a), the pelargonic acid and/or salt thereof (b), one or more than one additional antimicrobial agent (c), optionally one or more than one solvent (d) and optionally one or more than one excipient (e), wherein the one or more than one additional antimicrobial agent (c) is present in a minority amount relative to the combined amount of the antimicrobial agent (a) and the pelargonic acid and/or salt thereof (b), preferably wherein the one or more than one additional antimicrobial agent (c) is as described in Embodiment 10 above.
Embodiment 16: The antimicrobial composition of Embodiment 14 or 15, wherein the ratio by weight of the combined amount of (a) and (b) to the amount of the one or more than one additional antimicrobial agent (c) ranges from about 1.5:1 to about 150:1, such as from about 2:1, from about 3:1, from about 4:1, preferably from about 5:1, from about 9:1, more preferably from about 12:1, from about 16:1, particularly preferably from about 20:1 to about 150:1, preferably to about 100:1, more preferably to about 75:1 or to about 50:1.
Embodiment 17: The antimicrobial composition of any one of Embodiments 1-5, wherein the antimicrobial composition contains from about 70 to 100 wt %, from about 75 to 100 wt %, from about 80 to 100 wt %, from about 85 to 100 wt %, or from about 90 to 100 wt %, or preferably from about 70 wt %, more preferably from about 75 wt %, from about 80 wt % or from about 85 wt % to about 99 wt %, to about 97 wt %, to about 95 wt % or to about 92 wt % of the antimicrobial agent (a), the pelargonic acid and/or salt thereof (b), and one or more than one additional antimicrobial agent (c) combined, based on the total weight of the composition, preferably wherein the one or more than one additional antimicrobial agent (c) is as described in Embodiment 10 above.
Embodiment 18: The antimicrobial composition of any one of Embodiments 10-17, wherein the one or more than one additional antimicrobial agent (c) comprises an alkylhydroxamic acid and/or salt thereof, wherein the alkylhydroxamic acid and/or salt thereof contains at least one alkyl group of a chain length of from about 2 to about 22 carbon atoms, preferably from about 6 to about 12 carbon atoms, which may be branched or linear, preferably linear, substituted or unsubstituted, and saturated or unsaturated.
Embodiment 19, The antimicrobial composition of Embodiment 18, wherein the alkylhydroxamic acid and/or salt thereof is hexanohydroxamic acid, caprylhydroxamic acid, decanohydroxamic acid, laurohydroxamic acid or a salt thereof or a combination thereof, preferably caprylhydroxamic acid and/or a salt thereof.
Embodiment 20: The antimicrobial composition of any one of Embodiments 1-19, wherein the ratio by weight of the antimicrobial agent (a) to the pelargonic acid and/or salt thereof (b) is from about 15:1 to about 1:1.5, more preferably from about 10:1, even more preferably from about 5:1 or from about 3:1 to about 1:1.25, to about 1:1.2, to about 1:1.1, or more preferably to about 1:1.
Embodiment 21: The antimicrobial composition of any preceding Embodiment, further comprising one or more than one excipient (e) comprising at least one alkaline stabilizer.
Embodiment 22: The antimicrobial composition of Embodiment 21, wherein the antimicrobial composition has a pH of from about 5.0 to about 7.5, preferably from about 5.5 or from about 6.0 to about 7.5, to about 7.0 or to about 6.5.
Embodiment 23: The antimicrobial composition of Embodiment 21 or 22, wherein the at least one alkaline stabilizer comprises a base chosen from a metal (preferably an alkali metal or alkaline earth metal) hydroxide, oxide, carbonate, bicarbonate, phosphate, or hydride, an amine base or a combination thereof, preferably a metal hydroxide, and preferably wherein the metal is sodium, potassium, magnesium or calcium, and further preferably wherein the base of the alkaline stabilizer is present in an amount of from about 0.1 wt %, from about 0.3 wt %, more preferably from about 0.5 wt %, even more preferably from about 1 wt % or from about 3 wt % to about 10 wt %, preferably to about 8 wt % or to about 6 wt %, based on the total weight of the antimicrobial composition.
Embodiment 24: The antimicrobial composition of Embodiment 23, wherein the base is a metal hydroxide, preferably sodium hydroxide, potassium hydroxide or a combination thereof.
Embodiment 25: The antimicrobial composition of any one of Embodiments 21-24, wherein the antimicrobial composition exhibits≤5 wt % loss of the antimicrobial agent (a) and the pelargonic acid and/or salt thereof (b) combined when stored at 55° C. for 2 weeks, further preferably wherein the antimicrobial composition exhibits≤15 wt % loss, more preferably ≤10 wt % loss, or even more preferably ≤5 wt % loss, of the antimicrobial agent (a) and the pelargonic acid and/or salt thereof (b) combined when stored at 55° C. for 5 weeks.
Embodiment 26: An aqueous product formulation comprising an antimicrobial effective amount of the antimicrobial composition according to any one of Embodiments 1-25.
Embodiment 27: The aqueous product formulation of Embodiment 26, wherein the aqueous product formulation has a pH ranging from about 5.0, from about 5.5, from about 6.0 or from about 6.5 to about 12, to about 11, to about 10, to about 9.5, to about 9.0, to about 8.5, to about 8.0 or to about 7.5, or wherein the aqueous product formulation has a pH that is higher than 5.0, higher than 5.5, higher than 6.0, or higher than 6.5, preferably up to about 12, up to about 11, up to about 10, up to about 9, up to about 8.5, up to about 8.0 or up to about 7.5, or more preferably wherein the aqueous product formulation has a pH of at least 6.0, even more preferably higher than 6.0, such as a pH of at least 6.1, at least 6.3, most preferably at least 6.5, at least 6.7, at least 6.9, at least 7.0, at least 7.1, at least 7.2, at least 7.3, at least 7.4, or at least 7.5.
Embodiment 28: The aqueous product formulation of Embodiment 26 or 27 in the form of a personal care (e.g., cosmetic), home care, health care or industrial product formulation, preferably a personal care or home care formulation.
Embodiment 29: A personal care formulation comprising an antimicrobial effective amount of the antimicrobial composition according to any one of Embodiments 1-25, and one or more additional ingredients suitable for use in a personal care formulation.
Embodiment 30: A home care formulation comprising an antimicrobial effective amount of the antimicrobial composition according to any one of Embodiments 1-25, and one or more additional ingredients suitable for use in a home care formulation.
Embodiment 31: A method of controlling or inhibiting the growth of microorganisms in a product formulation, comprising adding to the formulation an antimicrobial effective amount of the antimicrobial composition according to any one of Embodiments 1-25, preferably wherein the formulation is a personal care (e.g., cosmetic), home care, health care or industrial product formulation, more preferably wherein the formulation is a personal care (e.g., cosmetic) or home care formulation.
Embodiment 32: The formulation or method of any one of Embodiments 26-31, wherein the antimicrobial effective amount of the antimicrobial composition provides from about 0.1 wt %, from about 0.3 wt % or from about 0.5 wt % to about 5 wt %, preferably to about 3 wt %, more preferably to about 2 wt %, to about 1.5 wt % or to about 1 wt %, of a combined amount of the antimicrobial agent (a), the pelargonic acid and/or salt thereof (b) and the optional one or more than one additional antimicrobial agent (c), based on the total weight of the formulation, preferably wherein the one or more than one additional antimicrobial agent (c) is as described in Embodiment 10 above.
Embodiment 33: The formulation or method of Embodiment 32, wherein the formulation contains no greater than 1 wt %, preferably no greater than 0.8 wt % or 0.7 wt %, more preferably no greater than 0.6 wt %, no greater than 0.5 wt %, no greater than 0.4 wt % or no greater than 0.3 wt % of the antimicrobial agent (a).
Embodiment 34: A method of enhancing the antimicrobial efficacy of an antimicrobial agent selected from the group consisting of phenoxyethanol, benzyl alcohol and combinations thereof, comprising combining or mixing pelargonic acid and/or a salt thereof with the antimicrobial agent in a ratio by weight of the antimicrobial agent to the pelargonic acid of from about 40:1 to about 1:5, preferably from about 40:1 or from about 20:1 to about 1:2.5, more preferably a ratio by weight as described in Embodiment 20.
Embodiment 35: The method of Embodiment 34, wherein the antimicrobial agent is used in a personal care (e.g., cosmetic), home care, health care or industrial product formulation, preferably a personal care or home care formulation, preferably wherein the formulation has a pH as described in Embodiment 27.
Embodiment 36: Use of pelargonic acid to enhance the antimicrobial efficacy of an antimicrobial agent selected from the group consisting of phenoxyethanol, benzyl alcohol and combinations thereof, wherein the ratio by weight of the antimicrobial agent to the pelargonic acid is from about 40:1 to about 1:5, preferably from about 40:1 or from about 20:1 to about 1:2.5, more preferably a ratio by weight as described in Embodiment 20.
Embodiment 37: The use of Embodiment 36, wherein the antimicrobial agent is used in a personal care (e.g., cosmetic), home care, health care or industrial product formulation, preferably a personal care or home care formulation, preferably wherein the formulation has a pH as described in Embodiment 27.
Embodiment 38: A method of reducing the minimum amount of an antimicrobial preservative needed for preservation activity in a product formulation, preferably a personal care or home care formulation, the method comprising combining or mixing pelargonic acid and/or a salt thereof with the antimicrobial agent in the product formulation, wherein the antimicrobial agent is selected from the group consisting of phenoxyethanol, benzyl alcohol and combinations thereof, and the ratio by weight of the antimicrobial agent to the pelargonic acid and/or salt thereof is from about 40:1 to about 1:5, preferably from about 40:1 or from about 20:1 to about 1:2.5, more preferably a ratio by weight as described in Embodiment 20.
Embodiment 39: The method of Embodiment 38, wherein the product formulation has a pH as described in Embodiment 27.

As used herein, the articles “a”, “an”, and “the” preceding an element or component of the invention are intended to be nonrestrictive regarding the number of instances (i.e., occurrences) of the element or component. Therefore “a”, “an”, and “the” should be read to include one or at least one, and the singular word form of the element or component also includes the plural unless the number is obviously meant to be singular.

As used herein, the term “comprising” means the presence of the stated features, integers, steps, or components and does not preclude the presence or addition of one or more other features, integers, steps, components or groups thereof. That is, the stated features, integers, steps, or components may include other features, integers, steps, components or groups thereof. The term “comprising” is intended to include embodiments encompassed by the terms “consisting essentially of” and “consisting of,” unless the context dictates otherwise.

The phrase “consisting of” limits the scope to which the phrase applies to the stated elements, steps, or ingredients, precluding the inclusion of other elements or materials, except for impurities ordinarily associated therewith. For example, in the context of the pelargonic acid of the present disclosure, such impurities may include, for example, unsaturation, branching, and/or varying chain lengths above or below nine carbons.

The phrase “consisting essentially of” limits the scope to which the phrase applies to the stated elements, steps, or ingredients and those that do not materially affect the basic and novel characteristic(s) of the claimed invention. “Consisting essentially of” occupies a middle ground between a closed scope that is written in a “consisting of” format and an open scope that is written in a “comprising” format.

As used herein, the term “about” modifying the quantity of an ingredient employed refers to variation in the numerical quantity that can occur, for example, through typical measuring and handling procedures used for making concentrates or use solutions in the real world; through inadvertent error in these procedures; through differences in the manufacture, source, or purity of the ingredients employed to make the compositions or carry out the methods; and the like.

Where present, all ranges are inclusive and combinable. For example, when a range of “1 to 5” is recited, the recited range should be construed as including ranges “1 to 4”, “1 to 3”, “1-2”, “1-2 & 4-5”, “1-3 & 5”, and the like.

When a parameter is given either as a range, preferred range, or a list of upper values and lower values, this is to be understood as specifically disclosing all ranges formed from any pair of any upper range limit or preferred value and any lower range limit or preferred value, regardless of whether ranges are separately disclosed. Where a range of numerical values is recited herein, unless otherwise stated, the range is intended to include the endpoints thereof, and all integers and fractions within the range.

### EXAMPLES

The following examples demonstrate surprising and synergistic enhancement of the antimicrobial activity of phenoxyethanol and benzyl alcohol when combined with pelargonic acid and such synergistic enhancement can be further improved by including an additional antimicrobial agent. The examples further show such unexpected enhancement in product formulations having a pH even well above the pKa of pelargonic acid. Pelargonic acid also exhibited chelating activity as a multifunctional property in contrast to 2-ethyl-hexyl glycerin, which showed no such activity.

### Example 1. Challenge Test Demonstrating the Antimicrobial Enhancement of Phenoxyethanol and Benzyl Alcohol when Combined with Pelargonic Acid

The preservative activity in representative rinse-off and leave-on formulations was evaluated using a modification of the Personal Care Products Council (PCPC—formerly the Cosmetic, Toiletry and Fragrance Association) protocol (Machtiger, et al. 2001. Determination of the Efficacy of Preservation of Non-Eye Area Water Miscible Cosmetic and Toiletry Formulations: Collaborative Study. J of AOAC International. 84:1, 101-109).

This evaluation was conducted using a four week, two or three-cycle (i.e., two-cycle for personal care and three-cycle for home care applications) microbial preservative efficacy test (i.e., challenge test) at 25° C. to determine the preservative efficacy of each treatment. Phenoxyethanol, benzyl alcohol and pelargonic acid were tested alone and in various combinations. The pelargonic acid used in the testing was purchased from Sigma-Aldrich. Combinations of phenoxyethanol or benzyl alcohol with benchmark multifunctional booster compounds were also tested for comparison. The preservative and booster ingredients were dosed and mixed thoroughly at room temperature into various formulations, specifically different personal care formulations including a lotion (leave-on, Table 1), a micellar water (rinse-off, Table 2), and a body wash formulation (rinse-off, Table 3) and different home care formulations including a manual dishwashing formulation (Table 4) and a liquid laundry detergent formulation (Table 5). In all cases after dosing, the pH of the final formulations were readjusted to match the pH of the original formulations prior to dosing, by using either citric acid, triethanolamine, or diluted sodium hydroxide.
Table 1 shows an exemplary formulation for carbomer-based lotion compositions. Carbomers are a commonly used synthetic rheological modifier in personal care products. The formulation below was made by heating the aqueous (A) and oil (B) phases separately to 75-80° C. The oil phase was then poured into the aqueous phase and mixed by a homogenizer at approximately 45° C. The formed oil-in-water emulsion was cooled to room temperature. Triethanolamine and/or diluted sodium hydroxide was used to adjust the final pH to 6.3 or 6.5 (as indicated in the examples below).

TABLE 1
Representative Lotion Formulation with Carbomer

| Phase | Ingredients | INCI Name | WT % |
|---|---|---|---|
| A | Water | Aqua | 88.8 |
| | Glycerin | Glycerin | 2.0 |
| | Carbopol 940 (Lubrizol) | Carbomer | 0.2 |
| B | Cream Maker CA-20 | Cetearyl Alcohol and | 2.0 |
| | | Ceteareth-20 | |
| | Rita GMS (Rita Corp) | Glyceryl Stearate | 1.0 |
| | Isopropyl Myristate | Isopropyl Myristate | 1.0 |
| | Shea Butter | Shea Butter | 1.0 |
| | Caprylic/Capric | Caprylic/Capric | 2.0 |
| | Triglyceride | Triglyceride | |
| | Isohexadecane | Isohexadecane | 1.0 |
| | Silicone Oil Xiameter | Dimethicone | 1.0 |
| | PMX-200 5 cSt | | |

Table 2 shows a representative micellar water formulation. The micellar water composition was made by adding all the ingredients into a beaker and heating the ingredient mixture to 75~80° C. The final product was cooled to room temperature and pH was adjusted to 6.0 (unless otherwise indicated in the examples below) with citric acid solution.

TABLE 2
Representative Micellar Water Composition

| Ingredients | INCI Name | WT % |
|---|---|---|
| Water | Aqua | 92.95 |
| Glycerin | Glycerin | 1.00 |
| Propylene glycol | Propylene glycol | 0.50 |
| Disodium EDTA | Disodium EDTA | 0.05 |
| Panthenol | Panthenol | 1.00 |
| Cremophor RH | PEG-40 Hydrogenated Castor Oil | 2.00 |
| EcoSense 3000 Surfactant | Decyl glucoside | 1.50 |
| AMISOFT LS-11 | Sodium lauroyl glutamate | 0.50 |
| Pluronic L64 | Poloxamer 184 | 0.50 |

Table 3 shows a representative body wash formulation. The composition was made by mixing ACULYN 88 and water in a beaker and then adding STEOL 230 and AMPHOSOL CA. The solution was mixed with an overhead mixer. Triethanolamine was used to adjust the final pH to 6.5 (unless otherwise indicated in the examples below).

TABLE 3
Representative Body Wash Formulation

| Ingredients | INCI Name | WT % |
|---|---|---|
| Water | Aqua | 70.5 |
| ACULYN 88 | Acrylates/steareth-20 methacrylate cross | 4 |
| | polymer | |
| STEOL 230 | sodium laureth sulfate (26% active), | 20 |
| | unpreserved | |
| AMPHOSOL | cocamidopropyl betaine (35% active), | 5.5 |
| CA | unpreserved | |

Table 4 shows a representative manual dishwashing formulation. The manual dishwashing formulation composition was made by adding all the ingredients into a beaker and heating the ingredient mixture to 60° C. The final product was cooled to room temperature and pH was adjusted to 5.5 with citric acid solution (unless otherwise indicated in the examples below).

TABLE 4
Representative Manual Dishwashing Formulation

| Ingredients | WT % |
|---|---|
| Water | 79.5 |
| Sodium lauryl ether sulfate | 6.0 |
| (70% aqueous solution) | |
| Sodium dodecylbenzenesulfonate | 12.0 |
| (50% aqueous solution) | |
| Cocomono Isopropanolamine | 2.0 |
| Sodium chloride | 0.5% |

Table 5 shows a representative liquid laundry detergent formulation. The Liquid Laundry Detergent 5 composition was made by adding all the ingredients into a beaker and heating the ingredient mixture to 40° C. The final product was cooled to room temperature and pH was adjusted to 7.5 with citric acid solution (unless otherwise indicated in the examples below).

TABLE 5
Liquid Laundry Detergent Formulation

| Ingredients | WT % |
|---|---|
| Water | 58.6 |
| Sodium lauryl ether sulfate | 21.0 |
| (70% aqueous solution) | |
| Cocamidopropyl betaine | 8.4 |
| (40% aqueous solution) | |
| Polyethoxylated tallow amine | 5.0 |
| Propylene glycol | 4.0 |
| C₁₂-C₁₅ fatty acid alcohol | 3.0 |
| ethoxylates | |

After dosing, the samples were divided into two separate five gram aliquots. One aliquot was inoculated with 50 μl of a diluted bacterial pool, and the second aliquot was inoculated with 50 μl of a diluted fungal pool as described below. Unpreserved product samples were included as a growth control.

For personal care products, a mixed bacterial inoculum was prepared using 24-hour cultures of the test bacteria (Table 6) grown in trypticase soy broth (TSB). For home care products, a mixed bacterial inoculum was prepared using 24-hour cultures of the test bacteria either from Table 6 (full pool) or Table 7 (simplified pool) grown in trypticase soy broth (TSB). Details are disclosed in the home care examples below. Equal volumes of the bacterial test strains were combined and diluted one to ten in phosphate buffer to obtain an inoculum of approximately 5×10⁷ to 5×10⁸ colony forming units per ml (cfu/ml). The test samples were inoculated with 1% of the mixed bacterial inoculum. For both personal care and home care products, a mixed fungal inoculum was prepared using cell suspensions of the yeast Candida albicans ATCC #10231 and the mold Aspergillus brasiliensis ATCC #16404 in phosphate buffer. Equal volumes of the fungal test strains were combined and diluted one to ten with phosphate buffer to obtain an inoculum of approximately 5×10⁶ to 5×10⁷ cfu/ml. The test samples were inoculated with 1% of the mixed fungal inoculum.

TABLE 6
Mixed Bacterial Inoculum for Personal Care and/or Home Care

| | ATCC | |
|---|---|---|
| Microorganism | Number | Type |
| Burkholderia cepacia | 25416 | Gram-Negative Non-fermenter |
| Enterobacter gergoviae | 33028 | Gram-Negative Fermenter |
| Escherichia coli | 8739 | Gram-Negative Fermenter |
| Klebsiella pneumoniae | 13883 | Gram-Negative Fermenter |
| Pseudomonas aeruginosa | 9027 | Gram-Negative Non-fermenter |
| Pseudomonas aeruginosa | 15442 | Gram-Negative Non-fermenter |
| Pseudomonas putida | 49128 | Gram-Negative Non-fermenter |
| Staphylococcus aureus | 6538 | Gram-Positive |
| Staphylococcus epidermidis | 12228 | Gram-Positive |

TABLE 7
Mixed Bacterial Inoculum for Home Care

| | ATCC | |
|---|---|---|
| Microorganism | Number | Type |
| Pseudomonas aeruginosa | 9027 | Gram-Negative Non-fermenter |
| Staphylococcus aureus | 6538 | Gram-Positive |

For personal care products, the samples were subjected to two microbial challenges at time zero and inoculated a second time after seven days. For home care products, the samples were subjected to three microbial challenges at time zero and inoculated a second and third time after seven and fourteen days, respectively. The number of microorganisms added to each sample was determined by a standard Most Probable Number (MPN) determination in Trypticase Soy Broth (TSB) for bacteria and Potato Dextrose Broth (PDB) for fungi. Samples were incubated at 25° C. for the test duration of four weeks. Samples were monitored for bacterial and fungal contamination after 2, 7, 14, 21, and 28 days. Samples challenged with bacteria were streak-plated onto Trypticase Soy Agar (TSA) and incubated at 30° C. for 24 hours. Samples challenged with fungi were streak-plated onto Potato Dextrose Agar (PDA) and incubated at 25° C. for 7 days. After incubation, plates were given a Growth Rating to determine the colony forming units per gram (CFU/g) present in each test sample at the testing time point.

Table 8 represents the growth score. As is used by various challenge test methods, the passing criteria is more stringent for bacteria than for fungi. A rating of 2, 3, or 4 at 21 and/or 28 days is considered a fail for bacteria. For fungi, a rating of 2 at 21 and/or 28 days is adequate and therefore a pass.

TABLE 8
Growth score used to determine CFU/mL in test samples.

| | Description of | | |
|---|---|---|---|
| Growth | Microbial | | Approximate |
| Rating | Growth | Plating Results | cfu/g |
| 0 | No contamination | No detectable | <10 |
| | | growth | |
| 1 | Trace contamination | 1 to 9 colonies | 1 × 10¹ to 9 × 10¹ |
| 2 | Light contamination | 10 to 99 colonies | 1 × 10² to 9.9 × 10² |
| 3 | Moderate | 100 to 1,000 | 1 × 10³ to 9.9 × 10³ |
| | contamination | colonies | |
| 4 | Heavy | >1,000 or smear | >1 × 10⁴ |
| | contamination | | |

For bacteria challenge testing, a composition was scored as “Pass” if the composition obtained a growth rating score caused by bacteria of 1 or less at days 21-28 of the testing period and was scored as “Fail” if the composition obtained a growth rating score caused by bacteria of 2 or more at days 21-28 of the testing period.

For fungi challenge testing, a composition was scored as “Pass” if the composition obtained a growth rating score caused by fungi of 2 or less at days 21-28 of the testing period and was scored as “Fail” if the composition obtained a growth rating score caused by fungi of more than 2 at days 21-28 of the testing period.

The “Pass” rating of a dosed formulation is also contingent upon the unpreserved sample having a rating of “Fail”.

### Example 1A

A representative leave-on lotion formulation was made according to Table 1 (pH 6.3). As shown in Table 9, while the lotion formulations dosed with the preservatives phenoxyethanol or benzyl alcohol alone at 0.4 wt % showed some antimicrobial activity compared to the unpreserved lotion (Blank), the formulations were far from passing the challenge test criteria. The formulations dosed with pelargonic acid alone also failed the challenge criteria. However, when the same dose of phenoxyethanol or benzyl alcohol was combined with pelargonic acid, the lotion formulations surprisingly showed significantly enhanced antimicrobial properties (i.e., ≥3 log reduction enhancement in terms of the colony forming units per gram (CFU/g) at day 21 or 28 for bacteria, and ≥1 log reduction enhancement at day 21 or 28 for fungi) against both bacteria and fungi, each achieving a passing rating in the challenge testing. This example demonstrates that pelargonic acid surprisingly enhances the efficacy of phenoxyethanol and benzyl alcohol, and advantageously so such that efficacious broad spectrum antimicrobial activity can be achieved even at low dosing levels of phenoxyethanol and benzyl alcohol (e.g., at 0.4 wt %).

TABLE 9
Challenge test data − lotion formulation comprising carbomer

| | Bacteria growth rating | | | Fungi growth rating | | | Overall |
|---|---|---|---|---|---|---|---|
| Sample and Dosing (wt %) | D21 | D28 | Result | D21 | D28 | Result | Result |
| Comparative − Lotion Blank | 4 | 4 | Fail | 4 | 4 | Fail | Fail |
| Comparative − Benzyl Alcohol (BA) (0.4%) | 3 | 3 | Fail | 4 | 4 | Fail | Fail |
| Comparative − Phenoxyethanol (POE) (0.4%) | 3 | 3 | Fail | 3 | 2 | Fail | Fail |
| Comparative − Pelargonic Acid (0.5%) | 4 | 4 | Fail | 3 | 3 | Fail | Fail |
| Comparative − Pelargonic Acid (1.0%) | 4 | 4 | Fail | 3 | 3 | Fail | Fail |
| POE (0.4%) + Pelargonic Acid (0.5%) | 0 | 0 | Pass | 2 | 2 | Pass | Pass |
| POE (0.4%) + Pelargonic Acid (1.0%) | 0 | 0 | Pass | 2 | 1 | Pass | Pass |
| BA (0.4%) + Pelargonic Acid (0.5%) | 0 | 0 | Pass | 2 | 1 | Pass | Pass |
| BA (0.4%) + Pelargonic Acid (1.0%) | 0 | 1 | Pass | 1 | 0 | Pass | Pass |

### Example 1B

A representative micellar water formulation was made according to Table 2 (pH 6.0). As shown in Table 10, the micellar water formulations passed the challenge test against bacteria when dosed with phenoxyethanol (POE) or benzyl alcohol (BA) alone, but neither phenoxyethanol nor benzyl alcohol alone was sufficient to protect the micellar water formulation against fungi growth. The micellar water formulations also failed the challenge test when dosed with pelargonic acid or with the commercial multifunctional boosters 2-ethyl-hexyl glycerin or 1,2 hexanediol alone.

In comparison, the micellar water formulation surprisingly showed significantly enhanced antimicrobial properties, particularly against fungi in this example, when the same dose of phenoxyethanol or benzyl alcohol was combined with pelargonic acid. Additionally, the combinations with pelargonic acid exhibited superior antimicrobial performance compared to the combinations with the two commercial multifunctional benchmarks 2-ethyl-hexyl glycerin (EHG) and 1,2 hexanediol. This example further demonstrates that pelargonic acid enhances the antimicrobial activity of phenoxyethanol and benzyl alcohol, and advantageously so such that efficacious broad spectrum antimicrobial activity can be achieved at reduced dosing levels of phenoxyethanol and benzyl alcohol (e.g., at 0.3 wt %).

TABLE 10
Challenge test data − micellar water formulation

| | Bacteria growth rating | | | Fungi growth rating | | | Overall |
|---|---|---|---|---|---|---|---|
| Sample and Dosing (wt %) | D21 | D28 | Result | D21 | D28 | Result | Result |
| Comparative − Micellar Water Blank | 4 | 4 | Fail | 4 | 3 | Fail | Fail |
| Comparative − Benzyl Alcohol (BA) (0.3%) | 0 | 0 | Pass | 4 | 3 | Fail | Fail |
| Comparative − Phenoxyethanol (POE) (0.3%) | 0 | 0 | Pass | 4 | 3 | Fail | Fail |
| Comparative − Pelargonic Acid (0.5%) | 4 | 4 | Fail | 3 | 3 | Fail | Fail |
| Comparative − 2-ethyl-hexyl glycerin | 4 | 4 | Fail | 4 | 4 | Fail | Fail |
| (EHG) (0.5%) | | | | | | | |
| Comparative − 1,2 hexanediol (0.5%) | 0 | 0 | Pass | 4 | 3 | Fail | Fail |
| BA (0.3%) + Pelargonic Acid (0.5%) | 0 | 0 | Pass | 0 | 0 | Pass | Pass |
| Comparative − BA (0.3%) + EHG (0.5%) | 0 | 0 | Pass | 0 | 1 | Pass | Pass |
| Comparative − BA (0.3%) + 1,2 hexanediol | 0 | 0 | Pass | 1 | 0 | Pass | Pass |
| (0.5%) | | | | | | | |
| POE (0.3%) + Pelargonic Acid (0.5%) | 0 | 0 | Pass | 1 | 1 | Pass | Pass |
| Comparative − POE (0.3%) + EHG (0.5%) | 0 | 0 | Pass | 3 | 3 | Fail | Fail |
| Comparative − POE (0.3%) + 1,2 hexanediol | 0 | 0 | Pass | 3 | 2 | Fail | Fail |
| (0.5%) | | | | | | | |

### Example 1C

A representative micellar water formulation was made according to Table 2 (pH 6.0). In this example, the inoculation strength for the bacteria pool was higher at ~10⁸ cfu/ml compared to the inoculation strength of ~10⁷ cfu/ml in Example 1B. As shown in Table 11, neither phenoxyethanol nor benzyl alcohol alone at a dosing level of 0.3 wt % adequately protected the micellar water formulation against either bacteria or fungi growth according to the challenge test criteria. As expected, the formulations dosed with pelargonic acid alone (0.3 wt %) failed the challenge testing. However, when the same dose of phenoxyethanol or benzyl alcohol was combined with pelargonic acid, the formulation unexpectedly showed significant antimicrobial enhancement to protect the formulation against both bacteria and fungi (an overall result of “Pass”). This example further demonstrates that pelargonic acid surprisingly enhances the antimicrobial efficacy of phenoxyethanol and benzyl alcohol such that efficacious broad spectrum antimicrobial activity can be achieved at reduced dosing levels of phenoxyethanol and benzyl alcohol (e.g., at 0.3 wt %).

TABLE 11
Challenge test data − micellar water formulation

| | Bacteria growth rating | | | Fungi growth rating | | | Overall |
|---|---|---|---|---|---|---|---|
| Sample and Dosing (wt %) | D21 | D28 | Result | D21 | D28 | Result | Result |
| Comparative − Micellar water Blank | 4 | 4 | Fail | 4 | 3 | Fail | Fail |
| Comparative − Benzyl Alcohol (BA) (0.3%) | 3 | 0 | Fail | 4 | 3 | Fail | Fail |
| Comparative − Phenoxyethanol (POE) (0.3%) | 2 | 1 | Fail | 3 | 3 | Fail | Fail |
| Comparative − Pelargonic Acid (0.3%) | 4 | 4 | Fail | 3 | 3 | Fail | Fail |
| BA (0.3%) + Pelargonic Acid (0.3%) | 0 | 0 | Pass | 2 | 2 | Pass | Pass |
| POE (0.3%) + Pelargonic Acid (0.3%) | 0 | 0 | Pass | 2 | 2 | Pass | Pass |

### Example 1D

An additional representative leave-on lotion formulation was made according to Table 1 (pH 6.3). Phenoxyethanol, benzyl alcohol and/or pelargonic acid were dosed as shown in Table 12 and the antibacterial challenge testing was conducted. Growth ratings at days 2, 7, 14, 21 and 28 are provided in Table 12. As shown, the lotion formulations did not pass the challenge test against bacteria when dosed with phenoxyethanol (POE, 0.4 wt %) or benzyl alcohol (BA, 0.4 wt %) alone. However, when the same dose of phenoxyethanol or benzyl alcohol was combined with pelargonic acid (0.4 wt %), the formulations passed the challenge test against bacteria. In addition, the combination 0.4% BA+0.4% pelargonic acid further unexpectedly showed superior antibacterial efficacy than benzyl alcohol alone at 0.8 wt % (e.g., 2 log reduction improvement at day 14 and 21). This example further demonstrates the synergistic enhancement of antimicrobial activity of benzyl alcohol when combined with pelargonic acid.

Furthermore, the combination 0.4% POE+0.4% pelargonic acid unexpectedly showed superior antibacterial efficacy than phenoxyethanol alone at 0.8 wt % (e.g., 1 log reduction improvement at day 2 and 3 log reduction improvement at day 7). This example further demonstrates the synergistic enhancement of antimicrobial activity of phenoxyethanol when combined with pelargonic acid.

TABLE 12
Challenge test data − lotion formulation comprising carbomer

| | Bacteria growth rating | | | | | |
|---|---|---|---|---|---|---|
| Sample and Dosing (wt %) | D2 | D7 | D14 | D21 | D28 | Result |
| Comparative − Lotion Blank | 4 | 4 | 4 | 4 | 4 | Fail |
| Comparative − Benzyl Alcohol | 4 | 4 | 4 | 4 | 2 | Fail |
| (BA) (0.4%) | | | | | | |
| Comparative − Benzyl Alcohol | 4 | 3 | 4 | 2 | 0 | Fail |
| (BA) (0.8%) | | | | | | |
| Comparative − Phenoxyethanol | 4 | 4 | 4 | 4 | 3 | Fail |
| (POE) (0.4%) | | | | | | |
| Comparative − Phenoxyethanol | 4 | 3 | 0 | 0 | 0 | Pass |
| (POE) (0.8%) | | | | | | |
| Comparative − Pelargonic Acid | 4 | 4 | 4 | 4 | 4 | Fail |
| (0.8%) | | | | | | |
| BA (0.4%) + Pelargonic Acid | 3 | 2 | 2 | 0 | 0 | Pass |
| (0.4%) | | | | | | |
| POE (0.4%) + Pelargonic Acid | 3 | 0 | 0 | 0 | 0 | Pass |
| (0.4%) | | | | | | |

### Example 1E

A representative body wash was made according to Table 3 (pH 6.5). As shown in Table 13, the unpreserved body wash formulation (Blank) passed the challenge criteria against fungi but failed against bacteria. Formulations dosed with benzyl alcohol (0.5 wt %) or pelargonic acid (0.5 wt %) alone also failed against bacteria. However, the body wash formulation dosed with benzyl alcohol (0.5 wt %) and pelargonic acid (0.5 wt %) surprisingly showed significant antimicrobial improvement, passing the challenge test. In a direct comparison, a combination of benzyl alcohol (0.5 wt %) and a benchmark multifunctional booster Ethylhexylglycerin (0.5 wt %) did not pass the challenge test and showed no improvement compared to the unpreserved body wash blank.

TABLE 13
Challenge test data − body wash

| | Bacteria growth rating | | | Fungi growth rating | | | Overall |
|---|---|---|---|---|---|---|---|
| Sample and Dosing (wt %) | D21 | D28 | Result | D21 | D28 | Result | Result |
| Comparative − Body Wash Blank | 4 | 4 | Fail | 2 | 2 | Pass | Fail |
| Comparative − Benzyl Alcohol (BA) (0.5%) | 4 | 4 | Fail | 2 | 1 | Pass | Fail |
| Comparative − Pelargonic Acid (0.5%) | 4 | 4 | Fail | 2 | 2 | Pass | Fail |
| Comparative − 2-ethyl-hexyl glycerin | 4 | 4 | Fail | 3 | 3 | Fail | Fail |
| (EHG) (0.5%) | | | | | | | |
| BA (0.5%) + Pelargonic Acid (0.5%) | 0 | 0 | Pass | 1 | 1 | Pass | Pass |
| Comparative − BA (0.5%) + | 4 | 4 | Fail | 2 | 2 | Pass | Fail |
| Ethylhexylglycerin (0.5%) | | | | | | | |

### Example 1F

A representative manual dishwashing formulation (pH=5.5) was made according to Table 4. A simplified bacteria pool (i.e., Table 7) was used for this example. As shown in Table 14, the antimicrobial agent phenoxyethanol alone dosed at 1.0 wt % failed the challenge testing. As expected, pelargonic acid alone dosed at 1.0 wt % also failed the challenge testing. However, combinations of phenoxyethanol and pelargonic acid at 50/50 (w/w) and at 60/40 (w/w) at the same total dosing level (1 wt %) showed significant improvement in antibacterial efficacy (i.e., 4 log reduction at day 21 compared to the phenoxyethanol alone and 4 log reduction at day 21 and 28 compared to pelargonic acid alone). The combinations also showed improvement in antifungal efficacy and was sufficient to pass the challenge test. This further demonstrates the synergistic enhancement of antimicrobial activity of phenoxyethanol when combined with pelargonic acid. Moreover, a 3-component combination with phenoxyethanol, pelargonic acid, and caprylhydroxamic acid (i.e., 49/49/2 and 59/39/2 w/w/w) at the same total dosing level (1 wt %) showed further improvement compared to the 2-component combinations. A dosing level of only 0.02 wt % of the caprylhydroxamic acid in these 3-component combinations was enough to deliver a 1-2 log reduction improvement at day 28 against fungi, compared to the 2-component combinations.

TABLE 14
Challenge test data − Manual Dishwashing

| | Bacteria growth rating | | | Fungi growth rating | | | Overall |
|---|---|---|---|---|---|---|---|
| Sample and Dosing (wt %) | D21 | D28 | Result | D21 | D28 | Result | Result |
| Comparative − Manual Dishwashing Blank | 4 | 4 | Fail | 3 | 3 | Fail | Fail |
| Comparative − Caprylhydroxamic acid (CHA) | 4 | 4 | Fail | 3 | 3 | Fail | Fail |
| (0.02%) | | | | | | | |
| Comparative − Phenoxyethanol (POE) (0.5%) | 4 | 4 | Fail | 3 | 3 | Fail | Fail |
| Comparative − Phenoxyethanol (POE) (1.0%) | 4 | 0 | Fail | 3 | 2 | Fail | Fail |
| Comparative − Pelargonic Acid (1.0%) | 4 | 4 | Fail | 3 | 3 | Fail | Fail |
| POE (0.5%) + Pelargonic Acid (0.5%) | 0 | 0 | Pass | 2 | 2 | Pass | Pass |
| POE (0.6%) + Pelargonic Acid (0.4%) | 0 | 0 | Pass | 2 | 2 | Pass | Pass |
| POE (0.49%) + Pelargonic Acid (0.49%) + | 0 | 0 | Pass | 2 | 1 | Pass | Pass |
| CHA (0.02%) | | | | | | | |
| POE (0.59%) + Pelargonic Acid (0.39%) + | 0 | 0 | Pass | 1 | 0 | Pass | Pass |
| CHA (0.02%) | | | | | | | |

### Example 1G

A representative liquid laundry detergent formulation (pH=7.5) was made according to Table 5. A simplified bacteria pool (i.e., Table 7) was used for this example. As shown in Table 15, phenoxyethanol alone at either 0.5% or 1.0% dosing level was not sufficient to pass the challenge test due to lack of antifungal efficacy, and pelargonic acid alone at 1.0% dosing level failed the challenge criteria against both bacteria and fungi. However, antimicrobial performance was synergistically enhanced by combinations of phenoxyethanol and pelargonic acid at 50/50 (w/w) and at 60/40 (w/w) at the same total dosing level of 1.0%. The combinations of phenoxyethanol and pelargonic acid showed significant improvement in antifungal efficacy compared to either phenoxyethanol or pelargonic acid alone at 1%. Moreover, a 3-component combination with phenoxyethanol, pelargonic acid, and caprylhydroxamic acid (at 49/49/2 and 59/39/2 w/w/w) showed additional surprising synergy and further improvement at a total dosing level of 1%, compared to either the 2-component combinations or each ingredient alone. A load level of only 0.02% of the caprylhydroxamic acid in these 3-component combinations was enough to deliver a 1 log reduction improvement at day 28 against fungi, compared to the 2-component combinations.

TABLE 15
Challenge test data − Liquid Washing

| | Bacteria growth rating | | | Fungi growth rating | | | Overall |
|---|---|---|---|---|---|---|---|
| Sample and Dosing (wt %) | D21 | D28 | Result | D21 | D28 | Result | Result |
| Comparative − Liquid Washing Blank | 4 | 4 | Fail | 3 | 2 | Fail | Fail |
| Comparative − Caprylhydroxamic acid (CHA) | 4 | 4 | Fail | 3 | 3 | Fail | Fail |
| (0.02%) | | | | | | | |
| Comparative − Phenoxyethanol (POE) (0.5%) | 1 | 0 | Pass | 3 | 3 | Fail | Fail |
| Comparative − Phenoxyethanol (POE) (1.0%) | 0 | 0 | Pass | 3 | 2 | Fail | Fail |
| Comparative − Pelargonic Acid (1.0%) | 4 | 4 | Fail | 3 | 2 | Fail | Fail |
| POE (0.5%) + Pelargonic Acid (0.5%) | 1 | 0 | Pass | 2 | 2 | Pass | Pass |
| POE (0.6%) + Pelargonic Acid (0.4%) | 0 | 0 | Pass | 2 | 1 | Pass | Pass |
| POE (0.49%) + Pelargonic Acid (0.49%) + | 0 | 0 | Pass | 2 | 1 | Pass | Pass |
| CHA (0.02%) | | | | | | | |
| POE (0.59%) + Pelargonic Acid (0.39%) + | 1 | 0 | Pass | 2 | 0 | Pass | Pass |
| CHA (0.02%) | | | | | | | |

### Example 1H

A representative liquid laundry detergent formulation (pH=7.5) was made according to Table 5. A full bacteria pool (i.e., Table 6) was used for this example (as compared to a simplified pool in Example 1G). As shown in Table 16, phenoxyethanol alone at either 0.5% or 1.0% dosing level was not sufficient to pass the challenge test due to lack of antifungal efficacy. With a full bacteria pool (i.e., Table 6), phenoxyethanol alone at 0.5% also failed against bacteria. Pelargonic acid alone at 1.0% dosing level failed the challenge criteria against both bacteria and fungi. However, antimicrobial performance was synergistically enhanced by combinations of phenoxyethanol and pelargonic acid at 60/40 (w/w) and 70/30 (w/w) at the same total dosing level of 1.0%. Moreover, a 3-component combination of phenoxyethanol, pelargonic acid, and caprylhydroxamic acid (at 59/39/2 and 69/29/2 w/w/w) showed additional surprising synergy and further improvement at a total dosing level of 1%, compared to either the 2-component combinations or each ingredient alone. A load level of only 0.02% of the caprylhydroxamic acid in these 3-component combinations was enough to deliver a 1 log reduction improvement at day 21 against fungi, compared to the 2-component combinations.

TABLE 16
Challenge test data − Liquid Washing

| | Bacteria growth rating | | | Fungi growth rating | | | Overall |
|---|---|---|---|---|---|---|---|
| Sample and Dosing (wt %) | D21 | D28 | Result | D21 | D28 | Result | Result |
| Comparative − Liquid Washing Blank | 4 | 4 | Fail | 3 | 3 | Fail | Fail |
| Comparative − Caprylhydroxamic acid (CHA) | 4 | 4 | Fail | 3 | 3 | Fail | Fail |
| (0.02%) | | | | | | | |
| Comparative − Phenoxyethanol (POE) (0.5%) | 4 | 4 | Fail | 3 | 3 | Fail | Fail |
| Comparative − Phenoxyethanol (POE) (1.0%) | 0 | 0 | Pass | 3 | 3 | Fail | Fail |
| Comparative − Pelargonic Acid (1.0%) | 4 | 4 | Fail | 3 | 1 | Fail | Fail |
| POE (0.6%) + Pelargonic Acid (0.4%) | 0 | 0 | Pass | 2 | 1 | Pass | Pass |
| POE (0.7%) + Pelargonic Acid (0.3%) | 0 | 0 | Pass | 2 | 1 | Pass | Pass |
| POE (0.59%) + Pelargonic Acid (0.39%) + | 0 | 0 | Pass | 1 | 1 | Pass | Pass |
| CHA (0.02%) | | | | | | | |
| POE (0.69%) + Pelargonic Acid (0.29%) + | 0 | 1 | Pass | 1 | 1 | Pass | Pass |
| CHA (0.02%) | | | | | | | |

### Example 1I

A representative leave-on lotion formulation (pH=6.5) was made according to Table 1. As shown in Table 17, phenoxyethanol alone at 0.4 wt % and pelargonic acid alone at 0.8 wt % failed the challenge criteria against bacteria and fungi. At a total dosing level of 0.8 wt %, the combinations of phenoxyethanol and pelargonic acid at various ratios (50/50, 60/40, 70/30, 80/20, 90/10 w/w) showed superior antifungal performance compared to phenoxyethanol at 0.8 wt % at day 2 (e.g., one log difference in the fungi growth rating). The combinations of phenoxyethanol and pelargonic acid additionally significantly improved antimicrobial performance compared to phenoxyethanol at 0.4 wt %, as particularly evidenced by the combination of phenoxyethanol and pelargonic acid at 50/50 w/w, which likewise applied phenoxyethanol at 0.4 wt %. The surprising performance improvement of the combination is attributed to the synergy between phenoxyethanol and pelargonic acid despite the pelargonic acid exhibiting little to no antimicrobial efficacy when used alone at the applied dosing levels. Furthermore, a 3-component combination of phenoxyethanol, pelargonic acid, and caprylhydroxamic acid (at 49/49/2, 69/29/2 and 69/29/2 w/w/w) showed additional surprising synergy and further improvement against both bacteria and fungi at a total dosing level of 0.8%, compared to either the 2-component combinations or each ingredient alone. A load level of only 0.016% of the caprylhydroxamic acid in these 3-component combinations was enough to deliver at least 1 log reduction improvement against fungi (day 2, day 21 and day 28) and at least 1 log reduction improvement against bacteria (at day 2), compared to the 2-component combinations.

TABLE 17
Challenge test data − lotion formulation comprising carbomer

| | Bacteria growth rating | | | | Fungi growth rating | | | | Overall |
|---|---|---|---|---|---|---|---|---|---|
| Sample and Dosing (wt %) | D2 | D21 | D28 | Result | D2 | D21 | D28 | Result | Result |
| Comparative − Lotion Blank | 4 | 4 | 4 | Fail | 4 | 4 | 4 | Fail | Fail |
| Comparative − Caprylhydroxamic | 4 | 4 | 4 | Fail | 4 | 4 | 4 | Fail | Fail |
| acid (CHA) (0.016%) | | | | | | | | | |
| Comparative − Phenoxyethanol (POE) | 4 | 3 | 1 | Fail | 4 | 3 | 2 | Fail | Fail |
| (0.4%) | | | | | | | | | |
| Comparative − Phenoxyethanol (POE) | 4 | 0 | 0 | Pass | 4 | 1 | 0 | Pass | Pass |
| (0.8%) | | | | | | | | | |
| Comparative − Pelargonic Acid (PA) | 4 | 4 | 4 | Fail | 4 | 4 | 4 | Fail | Fail |
| (0.8%) | | | | | | | | | |
| POE/PA (50/50 w/w) (0.8%) | 4 | 0 | 0 | Pass | 3 | 2 | 2 | Pass | Pass |
| POE/PA (60/40 w/w) (0.8%) | 3 | 0 | 0 | Pass | 3 | 1 | 1 | Pass | Pass |
| POE/PA (70/30 w/w) (0.8%) | 3 | 0 | 0 | Pass | 3 | 1 | 1 | Pass | Pass |
| POE/PA (80/20 w/w) (0.8%) | 3 | 0 | 0 | Pass | 3 | 1 | 1 | Pass | Pass |
| POE/PA (90/10 w/w) (0.8%) | 4 | 0 | 0 | Pass | 3 | 1 | 0 | Pass | Pass |
| POE/PA/CHA (49/49/2 w/w/w) | 1 | 0 | 0 | Pass | 2 | 0 | 0 | Pass | Pass |
| (0.8%) | | | | | | | | | |
| POE/PA/CHA (59/39/2 w/w/w) | 2 | 0 | 0 | Pass | 2 | 0 | 0 | Pass | Pass |
| (0.8%) | | | | | | | | | |
| POE/PA/CHA (69/29/2 w/w/w) | 0 | 0 | 0 | Pass | 2 | 0 | 0 | Pass | Pass |
| (0.8%) | | | | | | | | | |

### Example 2

The chelation property of the pelargonic acid was measured by using the ZenBio Cupric Ion Chelating (CIC) Assay (Cat #AOX-16). The assay measured the capacity of the pelargonic acid to chelate free cupric ions in solution, thereby inhibiting Cu(II) binding to pyrocatechol violet (PV) for generating a highly colored complex. The absorbance of the Cupric-PV complex was measured at a wavelength of 632 nm. The cupric ion chelating activity was determined as the percent of total PV over the Cu(II) binding. Ethylenediaminetetraacetic acid (EDTA), a well-known chelating agent, was used as a positive control. A series of EDTA with 7 different concentrations from 0 to 1000 μM were prepared and their corresponding absorbance measured and used as the calibration curve (Cupric ion chelating (%) vs. EDTA concentration). Percent cupric ion chelating was determined following the following calculations and Equation 1. The background absorbance value by averaging the absorbance values of the three background wells (no CuSO4 or sample). The average background absorbance was then subtracted from all the sample absorbance values. The maximal absorbance value (Absmax) was determined by averaging the absorbance values of the three wells containing CuSO4, PV and assay buffer. Abstest in Equation 1 is the absorbance of the test sample.

$$( Equation 1 )$$

$$Cupric ion chelating ( \% ) = 1 \; 00 \times ( Abs max - Abstest ) / Abs max$$

The calibration curve (Cupric ion chelating (%) vs. EDTA concentration) was fit by using a polynomial curve after log 10 transforming the concentrations (R2>0.98).

As shown in Table 18, Pelargonic Acid (775 μM) showed chelating activity that was equivalent to 667 μM EDTA. In contrast, ethylhexylglycerin (multifunctional) and phenoxyethanol (preservative) showed no chelating property (i.e., ~0 μM of EDTA equivalent) from the same assay.

TABLE 18
EDTA equivalent (calculated from a EDTA calibration curve)

| | | Calculated EDTA |
|---|---|---|
| Sample | Ingredient | equivalent (μM) |
| Sample 1 | Pelargonic Acid | 667 |
| Comparative 1 | Ethylhexylglycerin | ~0 |
| Comparative 2 | Phenoxyethanol | ~0 |

### Example 3

A series of preservative compositions with alkaline stabilizer were prepared according to Table 19. Phenoxyethanol and pelargonic acid were first mixed to form a homogenous clear solution. Sodium hydroxide (NaOH) or potassium hydroxide (KOH) was added to water to form an alkaline solution as the alkaline stabilizer. The alkaline solution was then slowly added to the homogenous clear solution. The pH values of the final compositions were measured (Table 19).

TABLE 19
Preservative compositions with alkaline stabilizer

| | Phenoxyeth- | Pelargonic | Alkaline Stabilizer | | |
|---|---|---|---|---|---|
| | anol | Acid | Base | Water | |
| Composition | (wt %) | (wt %) | (wt %) | (wt %) | pH |
| Comparative 1 | 66.67 | 33.33 | 0 | 0 | 2.6 |
| Comparative 2 | 60 | 30 | 0 | 10 | 3.1 |
| Sample 1 | 60 | 30 | NaOH (2.5) | 7.5 | 6.1 |
| Sample 2 | 60 | 30 | KOH (3) | 7 | 6.1 |
| Sample 3 | 60 | 30 | KOH (1) | 9 | 5.1 |
| Sample 4 | 60 | 30 | KOH (4) | 6 | 6.5 |

All preservative compositions were stored in an oven at a temperature of 55° C. Gas chromatography (GC) was used to measure the wt % loss of phenoxyethanol (POE) and pelargonic acid (PA) combined after 2 weeks and 5 weeks. As shown in Table 20, the addition of the alkaline solution stabilized the composition at elevated temperature over time (i.e., the compositions showed much lower wt % loss over time). In particular, Samples 2 and 4 showed excellent shelf-life stability with less than 4.0 wt % loss of phenoxyethanol and pelargonic acid combined when stored at 55° C. for 5 weeks.

TABLE 20
Preservative composition stability with alkaline stabilizer

| | Week 2 | Week 5 |
|---|---|---|
| | Combined loss | Combined loss |
| | of POE and PA | of POE and PA |
| Composition | (wt %) | (wt %) |
| Comparative 1 | 13 | 21 |
| Comparative 2 | 7.2 | 23 |
| Sample 1 | 4.5 | 9.6 |
| Sample 2 | <4.0 | <4.0 |
| Sample 3 | <4.0 | 7.1 |
| Sample 4 | <4.0 | <4.0 |

### Example 4

A representative leave-on lotion formulation was made according to Table 1 (pH 6.5). As shown in Table 21, the preservative composition comprising phenoxyethanol (POE), pelargonic acid (PA) and an alkaline stabilizer which was an alkaline solution of potassium hydroxide (KOH+H₂O) showed better efficacy against fungi compared to phenoxyethanol alone (the preservative composition also used 40% less POE compared to the sample dosed with POE alone). Further, as shown in Table 21, the preservative composition comprising POE, PA, the alkaline stabilizer and caprylhydroxamic acid (CHA) showed additional synergy and improvement at the same total dosing level (0.8%) compared to phenoxyethanol alone, even where the CHA load level was as low as 0.016% of the lotion formulation.

TABLE 21
Challenge test data − lotion formulation

| | Fungi growth rating | | | | | |
|---|---|---|---|---|---|---|
| Sample and Dosing (wt %) | D2 | D7 | D14 | D21 | D28 | Result |
| Comparative − Lotion Blank | 4 | 4 | 4 | 4 | 4 | Fail |
| Comparative − Phenoxyethanol (POE) (0.8%) | 3 | 2 | 2 | 2 | 2 | Pass |
| Sample 2 from Table 19 | 2 | 2 | 2 | 2 | 2 | Pass |
| (POE/PA/KOH/H₂O (60/30/3/7 w/w/w/w)) (0.8%) | | | | | | |
| POE/PA/CHA/KOH/H₂O (59/29/2/3/7 w/w/w/w) (0.8%) | 2 | 0 | 0 | 0 | 0 | Pass |

A representative manual dishwash formulation was made according to Table 4 (pH 6.5). The dishwash formulation was dosed with phenoxyethanol (POE) alone at a load level of 0.8 wt % and, separately, the dishwash formulation was dosed with the same preservative compositions as shown in Table 21 above, each at a total load level of 0.8 wt %. As shown in Table 22, the preservative compositions of the present disclosure showed better efficacy against fungi compared to phenoxyethanol alone. The manual dishwash formulation dosed with the preservative blends passed the challenge test against fungi with significant efficacy improvement (e.g., >2 log reduction improvement at day 21 and 28).

TABLE 22
Challenge test data − Manual dishwash formulation

| | Fungi growth rating | | | | | |
|---|---|---|---|---|---|---|
| Sample and Dosing (wt %) | D2 | D7 | D14 | D21 | D28 | Result |
| Comparative − Manual dishwash Blank | 3 | 2 | 3 | 3 | 3 | Fail |
| Comparative − Phenoxyethanol (POE) (0.8%) | 3 | 1 | 2 | 3 | 2 | Fail |
| Sample 2 from Table 19 | 2 | 0 | 0 | 1 | 0 | Pass |
| (POE/PA/KOH/H₂O (60/30/3/7 w/w/w/w)) (0.8%) | | | | | | |
| POE/PA/CHA/KOH/H₂O (59/29/2/3/7 w/w/w/w) (0.8%) | 2 | 0 | 0 | 0 | 0 | Pass |

1-44. (canceled)

45. A process for preparing an antimicrobial composition comprising
combining (a) an antimicrobial agent selected from the group consisting of phenoxyethanol, benzyl alcohol and combinations thereof and (b) pelargonic acid, in an amount of the components (a) and (b) combined of at least 50 wt %, based on the total weight of all ingredients combined to prepare the composition, and in a ratio by weight of the antimicrobial agent (a) to the pelargonic acid (b) of from about 20:1 to about 1:2.5, and (c) an alkaline stabilizer in an amount of from about 0.2 wt % to about 25 wt %, based on the total weight of all ingredients combined to prepare the composition, wherein the alkaline stabilizer is in the form of an alkaline aqueous solution, and the composition has a pH of from about 5.0 to about 7.5.

46. The process of claim 45, wherein the antimicrobial agent (a) is phenoxyethanol.

47. The process of claim 45, wherein the alkaline stabilizer comprises a base that is at least one metal hydroxide.

48. The process of claim 47, wherein the at least one metal hydroxide is chosen from sodium hydroxide, potassium hydroxide or a combination thereof.

49. An antimicrobial composition comprising
(a) an antimicrobial agent selected from the group consisting of phenoxyethanol, benzyl alcohol and combinations thereof, and (b) pelargonic acid and/or a salt thereof, wherein the antimicrobial agent (a) and the pelargonic acid and/or salt thereof (b) are present in a combined amount of at least 50 wt %, based on the total weight of the composition, and the ratio by weight of the antimicrobial agent (a) to the pelargonic acid and/or salt thereof (b) is from about 20:1 to about 1:2.5, and
(c) an alkylhydroxamic acid and/or a salt thereof in an amount no greater than 10 wt %, based on the total weight of the composition.

50. The antimicrobial composition of claim 49, wherein the antimicrobial agent (a) is phenoxyethanol.

51. The antimicrobial composition of claim 49, wherein the antimicrobial composition comprises the alkylhydroxamic acid and/or salt thereof (c) in an amount no greater than 5 wt %, based on the total weight of the antimicrobial composition.

52. The antimicrobial composition of claim 49, wherein the alkylhydroxamic acid and/or salt thereof is caprylhydroxamic acid and/or a salt thereof.

53. A personal care or home care product formulation comprising
an antimicrobial effective amount of a combination of (a) an antimicrobial agent selected from the group consisting of phenoxyethanol, benzyl alcohol and combinations thereof, and (b) pelargonic acid and/or a salt thereof, and
one or more additional ingredients suitable for use in a personal care or home care product formulation, wherein the ratio by weight of the antimicrobial agent (a) to the pelargonic acid and/or salt thereof (b) is from about 20:1 to about 1:2.5,
the product formulation has a pH of at least 6.0, and
the antimicrobial agent (a) constitutes no greater than 0.7 wt %, based on the total weight of the product formulation.

54. The product formulation of claim 53, wherein the antimicrobial agent (a) is phenoxyethanol.

55. A method of controlling microbial growth in or preserving a product formulation, comprising
adding to the product formulation (a) an antimicrobial agent selected from the group consisting of phenoxyethanol, benzyl alcohol and combinations thereof, and (b) pelargonic acid and/or a salt thereof, wherein the antimicrobial agent (a) and the pelargonic acid and/or salt thereof (b) are present in the formulation in a combined antimicrobial effective amount,
the ratio by weight of the antimicrobial agent (a) to the pelargonic acid and/or salt thereof (b) is from about 20:1 to about 1:2.5,
the product formulation has a pH of at least 6.0, and
the antimicrobial agent (a) constitutes no greater than 0.7 wt %, based on the total weight of the product formulation.

56. The method of claim 55, wherein the antimicrobial agent (a) is phenoxyethanol.

57. A personal care or home care product formulation comprising
an antimicrobial effective amount of from about 0.2 wt % to about 3 wt %, based on the total weight of the formulation, of a combination of (a) an antimicrobial agent selected from the group consisting of phenoxyethanol, benzyl alcohol and combinations thereof and (b) pelargonic acid and/or a salt thereof,
(c) an alkylhydroxamic acid and/or a salt thereof, and
one or more additional ingredients suitable for use in a personal care or home care formulation,
wherein the ratio by weight of the antimicrobial agent (a) to the pelargonic acid and/or salt thereof (b) is from about 20:1 to about 1:2.5, and the ratio by weight of the combined amount of (a) and (b) to the amount of the alkylhydroxamic acid and/or salt thereof (c) is from about 5:1 to about 150:1.

58. The product formulation of claim 57, wherein the antimicrobial agent (a) is phenoxyethanol.

59. The product formulation of claim 57, wherein the alkylhydroxamic acid and/or salt thereof is caprylhydroxamic acid and/or a salt thereof.

60. The product formulation of claim 57, wherein the product formulation has a pH of at least 6.0.

61. A method of controlling microbial growth in or preserving a product formulation, comprising
adding to the product formulation (a) an antimicrobial agent selected from the group consisting of phenoxyethanol, benzyl alcohol and combinations thereof, (b) pelargonic acid and/or a salt thereof, and (c) an alkylhydroxamic acid and/or a salt thereof,
wherein the antimicrobial agent (a) and the pelargonic acid and/or salt thereof (b) are present in the formulation in a combined antimicrobial effective amount of from about 0.2 wt % to about 3 wt %, based on the total weight of the formulation, and
wherein the ratio by weight of the antimicrobial agent (a) to the pelargonic acid and/or salt thereof (b) is from about 20:1 to about 1:2.5, and the ratio by weight of the combined amount of (a) and (b) to the amount of the alkylhydroxamic acid and/or salt thereof (c) is from about 5:1 to about 150:1.

62. The method of claim 61, wherein the antimicrobial agent (a) is phenoxyethanol.

63. The method of claim 61, wherein the alkylhydroxamic acid and/or salt thereof is caprylhydroxamic acid and/or a salt thereof.

64. The method of claim 61, wherein the product formulation has a pH of at least 6.0.
